(19) United States
(12) Patent Application Publication
(10) Pub. No.: US 2023/0295560 A1
(43) Pub. Date: Sep. 21, 2023

(54) SYNTHETIC ECOLOGIES FOR DRUG DISCOVERY

(71) Applicant: William Marsh Rice University, Houston, TX (US)

(72) Inventors: Yousif SHAMOO, Houston, TX (US); Heer MEHTA, Houston, TX (US); Ramya Ganiga PRABHAKAR, Bangalore (IN); Seokju SEO, Houston, TX (US)

(73) Assignee: William Marsh Rice University, Houston, TX (US)

(21) Appl. No.: 18/054,714

(22) Filed: Nov. 11, 2022

Related U.S. Application Data
(60) Provisional application No. 63/278,354, filed on Nov. 11, 2021.

(51) Int. Cl. C12N 1/20; C12N 1/14; C12M 3/06

(52) U.S. Cl. CPC C12N 1/205; C12N 1/145; C12M 23/16; C12R 2001/465

(57) ABSTRACT

The present disclosure is directed to composition and methods for use in drug screening methods. The identification of microbial strains producing antibiotics effective against multi-drug resistant bacterial pathogens using microfluidics and co-encapsulation of predator and prey strains permits rapid and multiplexed assessment of new antibiotics for emerging bacterial pathogens including those resistant to multiple existing antibiotics.

### PRIORITY CLAIM

This application claims benefit of priority to U.S. Provisional Application Ser. No. 63/278,354, filed Nov. 11, 2021, the entire contents of which are hereby incorporated by reference.

### STATEMENT REGARDING FEDERALLY SPONSORED RESEARCH

This invention was made with government support under Grant No. W81XWH1910679 awarded by the Department of Defense. The government has certain rights in the invention.

### BACKGROUND

### 1. Field of the Disclosure

The present disclosure relates generally to the fields of microbiology and drug discovery. More particular, the disclosure relates to rapid methods for the identification of microbial strains producing antibiotics effective against multi-drug resistant bacterial pathogens.

### 2. Background

The rise of Multi-Drug Resistant (MDRs) pathogens is one of the most pressing biomedical problems of this century for civilians and warfighters alikel^{1, 2}. As multi-drug resistance spreads, infectious disease physicians are increasingly forced to use drugs-of-last-resort. Unaddressed, the ascent of pan-resistant strains would bring a ‘post-antibiotic’ era in which all modern medicine would be threatened³⁻⁵.

‘ESKAPE’ pathogens (Enterococcus faecium, Staphylococcus aureus, Klebsiella pneumoniae, Acinetobacter baumannii, Pseudomonas aeruginosa, Enterobacter spp.) are associated with U.S. hospital-acquired infections for which new antibiotics are urgently needed^{6,7}. In addition, military infectious disease challenges not only range from the known ESKAPE pathogens within the military medical system but also to emerging pathogens or WMDs that might be encountered by servicemen and women in theater. Thus, there remains an urgent need to address two important questions that relate directly to these challenges. First, what can be done to reliably and programmatically discover new antibiotics or strategies to combat infection? And second, how can researchers react to emergent threat organisms in a timely fashion. Providing the answers to these questions would present a significant step forward in the development and deployment of new therapeutic strategies for bacterial infections in general, and in specific for drug-resistant strains.

### SUMMARY

Thus, in accordance with the present disclosure, there is provided microenvironment comprising a single predator strain of an antibiotic-producing microbe and a single strain of a multi-drug resistant (MDR) bacterial pathogen. The microenvironment may be a water-in-oil (W/O) microdroplet, such as wherein the microdroplet (a) is about 10-200 μm in diameter or about 100 μm in diameter; and/or (b) comprises 10 or fewer than microbial cells, such as 1 or 2 predator strain cells and 1 to 10 MDR pathogen cells. The microenvironment is defined as not encompassing a natural cell of any kind, i.e., is a non-natural, engineered and/or man-made microenvironment.

The predator strain may produce a fluorescent signal and/or the MDR bacterial pathogen produces a fluorescent signal. The predator strain may produce a first fluorescent signal and the MDR bacterial pathogen may produce a second fluorescent signal, the first and second fluorescent signals being optically distinguishable. The predator strain may be a culturable bacterium is selected from the group of wild or cultured isolates belonging to the genus Streptomyces, Bacillus, or Nocardia or a fungus belonging to the genus Penicillium. The MDR bacterial pathogen may be selected from the group consisting of Pseudomonas, Nocardia, Escherichia, Klebsiella, Staphylococcus, Acinetobacter, Francisella, or Enterococcus.

The microenvironment may be disposed in a bacterial growth chamber containing bacterial growth media. The bacterial growth chamber may comprise additional microenvironments each comprising the predator strain and the MDR bacterial pathogen. The predator strain may have been subjected to a mutagen, such as N-methyl-N′-nitro-N-nitrosoguanidine.

Also provided is a method of co-culturing a single predator strain of an antibiotic-producing microbe and a single strain of a multi-drug resistant (MDR) bacterial pathogen comprising (a) microencapsulating a single predator strain of an antibiotic-producing microbe and a single strain of a MDR bacterial pathogen to create a microenvironment; and (b) culturing the microenvironment in a bacterial growth chamber containing bacterial growth medium. The microenvironment may be a water-in-oil (W/O) microdroplet, such as generated by mixing aqueous and oil phases, in particular being generated through microfluidic processing. The microdroplet may (a) be about 10-200 μm in diameter or about 100 μm in diameter; and/or (b) comprise 10 or fewer than microbial cells, such as 1 or 2 predator strain cells and 1 or 2 MDR pathogen cells.

The predator strain may produce a fluorescent signal and/or the MDR bacterial pathogen produces a fluorescent signal. The predator strain may produce a first fluorescent signal and the MDR bacterial pathogen may produce a second fluorescent signal, the first and second fluorescent signals being optically distinguishable. The method may further comprise assessing the relative amounts of the predator strain and the MDR bacterial pathogen after culture. The method may further comprise isolating the microenvironment when the relative amount of predator strain present is greater than the MDR bacterial pathogen, such as by fluorescence activated sorting. The method may further comprise obtaining the predator strain from the isolated microenvironment.

The predator strain may be a culturable bacterium is selected from the group wild or cultured isolates belonging to the genus Streptomyces, Bacillus, or Nocardia or a fungus belonging to the genus Penicillium. The MDR bacterial pathogen may be selected from the group consisting of Pseudomonas, Nocardia, Escherichia, Klebsiella, Staphylococcus, Acinetobacter, Francisella, or Enterococcus. The bacterial growth chamber may comprise additional microenvironments each comprising the predator strain and the MDR bacterial pathogen. The predator strain may have been subjected to a mutagen. The culturing may be performed for about 24 hours to about 72 hours, or for about 48 hours, optionally at 30° C.

The method may further comprise (c) decapsulating the selected predator strain; (d) re-microencapsulating the selected predator strain and a single strain of a MDR bacterial pathogen to create a second microenvironment; (e) culturing the second microenvironment in a bacterial growth chamber containing bacterial growth medium; (f) assessing the relative amounts of the predator strain and the MDR bacterial pathogen of step (d) after culture; (g) isolating the second microenvironment when the relative amount of predator strain present is greater than the MDR bacterial pathogen; and (h) obtaining the predator strain from the isolated second microenvironment. This method may be further repeated, such as for a total of 10 cycles. A further mutagenesis step may applied to the predator strain after one or more cycles. The method may further comprise changing microenvironment volume and/or ratio of the predator strain to the MDR bacterial pathogen between cycles.

Step (b) may be performed using a microfluidic system employing flow-focusing geometry. Assessing may be performed using a microfluidic system employing flow-focusing geometry. Isolating may be performed using a microfluidic system employing flow-focusing geometry.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Microfluidic devices fabricated for this project (left) and an example of Predator S. venezuelae^{RFP} (red) and the Prey—P. aeruginosa^{GFP} (green) co-encapsulated in 100 μm microdroplets (right).

FIG. 2. Cartoon illustrating how a MASEDD Predator-Prey “cage match” selects for variants with increased ability to inhibit Prey cells. The potential Predator strains are Streptomyces spp. (pink) while the Prey is a pathogen (green). Potential Predators and Prey are co-encapsulated in microdroplets. (a) In most cases, the two strains simply compete for resources, e.g., the pink strain does not directly inhibit the green cells (an indifferent pair). (b), Using MASEDD, mutations in the pink strain have been selected for (now red) that allow it to inhibit growth of the green cell (an exploitative pair). Any culturable strain can be used as the potential candidate Predator or Prey.

FIG. 3. Schematic of the MASEDD selection. Initially, the inventors mutagenize S. venezuelae^{RFP} (Predator candidates) to increase mutation supply and then co-encapsulate with colistin resistant P. aeruginosa^{GFP} (Prey). They then select and enrich for variants of S. venezuelae^{RFP} that inhibit growth of P. aeruginosa^{GFP} more strongly than the ancestor. Using Fluorescence Activated Droplet Sorting (FADS), the inventors gate to collect the microdroplets with weaker GFP signal as a proxy for improved S. venezuelae^{RFP} ability to inhibit P. aeruginosa^{GFP}. The recaptured S. venezuelae^{RFP} will be used for the next iteration with fresh P. aeruginosa^{GFP}.

FIG. 4. Basic technology schematic of the MASEDD process using the Predator-Prey concept described in FIG. 2. The microfluidic devices can encapsulate 2.6 million individual Predator-Prey cage matches within each round of MASEDD in less than 30 min (DropletGen). After allowing the cells within the microdroplets to compete, the inventors use fluorescence activated droplets sorting (FADS) to isolate microdroplets in which the Predators have increased their ability to inhibit the Prey.

FIGS. 5A-B. Demonstration of co-encapsulation technology using DropletGen, GrowthChamber and FADS technologies. After 24 hours of growth at 30° C. in the GrowthChamber P. aeruginosa^{GFP} and S. venezuelae^{RFP} grow relatively independently within a single microdroplet as indicated by the yellow/orange merged image (FIG. 5A). If this microdroplet had contained a variant of S. venezuelae^{RFP} with the ability to inhibit P. aeruginosa^{GFP}, the microdroplet would be dark orange or red indicating increased success by S. venezuelae^{RFP} (Predator) in inhibiting P. aeruginosa^{GFP} (Prey) (FIG. 5B).

FIGS. 6A-B. Design of microfluidic devices. (FIG. 6A) Microfluidic device for Predator (P. aeruginosa^{GFP}) and Prey (S. venezuelae^{RFP}) co-encapsulation. The two aqueous streams are completely mixed after encapsulation into droplets from the laminar flow within the microchannels. (FIG. 6B) Microfluidic device for FADS. When incubated droplets pass a 488-nm laser slit, the resulting fluorescence is recorded, the sorting electric field is turned on only if the droplet has a fluorescence intensity above a preselected user-set threshold (“Sorting Threshold” in FIG. 3).

FIGS. 7A-B. S. venezuelae^{RFP} isolates from MASEDD inhibit P. aeruginosa^{GFP} as expected. Co-cultures of cells were grown in a 96-well plate format. (FIG. 7B) Control: Co-culture of original (S. venezuelae^{RFP}+P aeruginosa^{GFP}). S. venezuelae^{RFP} (red line) shows no ability to inhibit the growth of P. aeruginosa^{GFP} (green line); (FIG. 7B) Co-culture of (S. venezuelae^{RFP} from MASEDD+P. aeruginosa^{GFP}) S. venezuelae^{RFP} from MASEDD dramatically inhibits growth of P. aeruginosa^{GFP} (green line).

FIG. 8. The inventors have built high throughput and tunable droplet generators devices for co-encapsulation of cells. Representative optical microscopy images of microdroplet production from 50-200 μm (upper). Quality of microdroplet production as determined by histograms of measured diameters from optical microscopy images and fitted using the Gaussian or Lorentzian functions (lower).

FIG. 9. Encapsulated S. venezuelae^{RFP} growing as mycelial mats within microdroplets.

FIG. 10. P. aeruginosa PAO1 chromosomally labelled with GFP. Fluorescence is observed under blue light with an orange filter. Wild-type PAO1 does not fluoresce under these conditions.

FIGS. 11A-B. Conditioned Media Assay to determine lead efficacy against ESKAPE pathogens. Conditioned media is added at varying concentrations to fresh growth media inoculated with an ESKAPE pathogen: No conditioned media control (Blue), 2.5% (Red), 5% (Green), 10% (Purple). (FIG. 11A) ESKAPE pathogen P. aeurginosa PAO1; (FIG. 11B) MRSA. The molecule(s) produced by Streptomyces T4-11 (a wild isolate) do not inhibit P. aeurginosa PAO1 but shows activity against Gram-positive MRSA 131.

FIGS. 12A-B. Representative fluorescence microscopy images of 93 μm microdroplets containing co-encapsulations of (FIG. 12A) the starting strain of S. venezuelae^{RFP} and P. aeruginosa^{GFP} and (FIG. 12B) S. venezuelae^{RFP} MASEDD variants and P. aeruginosa^{GFP}. X is the average number of each strain in a given microdroplet.

FIGS. 13A-C. S. venezuelae^{RFP} isolate D from MASEDD inhibits P. aeruginosa^{GFP} as expected. Co-cultures of cells were grown in a 96-well plate format. a) Control: Co-cultures of the original S. venezuelae^{RFP} with different cell ratios (1:0, 1:20, 1:36, and 1:60) of S. venezuelae^{RFP} to P. aeruginosa^{GFP}. As shown in FIG. 13A the original S. venezuelae^{RFP} strain (red line) and P. aeruginosa^{GFP} (green line) grow well together with no strong inhibition; (FIG. 13B) Co-culture of (Isolate DRFP from MASEDD+P. aeruginosa^{GFP}) S. venezuelae^{RFP} from MASEDD strongly inhibits the growth of P. aeruginosa^{GFP} (green line). (FIG. 13C) Control: P. aeruginosa^{GFP} alone.

FIGS. 14A-B. Spent media from S. venezuelae^{RFP} isolate D culture inhibits growth of P. aeruginosa^{GFP} and E. coli^{GFP}. After incubating a monoculture of S. venezuelae^{RFP} isolate D for 2-days, cells were removed by centrifugation and filtration and the spent media was lyophilized to make a 20× concentrated crude preparation for further characterization. The concentrated spent media was mixed with fresh media in different v/v ratios (0-12.5%). (FIG. 14A) P. aeruginosa^{GFP} and (FIG. 14B) E. coli^{GFP} were inoculated with spent media and dose-dependent inhibitory activity was observed as expected.

FIGS. 15A-E. Flow-focusing geometry microdroplet generators rapidly produce highly homogenous microdroplets suitable for experimental evolution. (FIG. 15A) Design of a flow-focusing geometry. Representative microscopy images of flow-focusing geometries (top) and observation chambers (bottom) for (FIG. 15B) 93±2.3 μm, (FIG. 15C) 153±3.9 μm, and (FIG. 15D) 247±3.5 μm microdroplets. (FIG. 15E) Histograms of microdroplet diameters estimated using Gaussian fittings of microdroplets produced from flow-focused generators with optimized flow rates of dispersed water (W) and continuous oil (0) phases.

FIGS. 16A-F. Microemulsion studies and population growth as a function of the DOX selection gradient. (FIG. 16A) Schematic for serial-transfer of E. coli in the presence of DOX in microemulsions: The cells are diluted in fresh media before inoculating into new emulsion droplets to maintain a starting population size of ˜2.6×10⁶ cells/ml and increasing DOX concentrations. (FIGS. 16B-F) DOX concentration gradients and daily population sizes from microdroplet evolved populations. The DOX gradient is indicated as shaded areas and accompanying concentrations on the right y-axis. Average growth with each iteration is indicated as lines and accompanying OD on the left y-axis. Error bars represent the standard deviation for three populations and the red line indicates the MIC of DOX for the ancestor (4 μg/ml).

FIG. 17. Venn diagram showing mutations implicated in resistance identified in final flask evolved and microdroplet evolved populations. Major genetic drivers to DOX resistance (acrR and marR) were identified by both methods. While some of the adaptive changes within specific genes were commonly identified using both methods, some of the mutations were unique to one of the two. Particularly interesting were the genomic re-arrangements involving amplification events that were seen only in the microdroplet evolved populations.

FIG. 18. Heat map showing frequency of the occurrence of a mutant allele within populations evolved using the 6 experimental conditions shown in Table 2. Genes implicated in resistance to DOX from previous studies have been shown here. Gradient indicates the percentage of total replicate populations containing a particular mutation. Data was collected from 5 replicate populations for flask F, D1, D2 and D3, 2 surviving replicate populations for D4 and 3 replicate populations for D5. It is evident that while a larger number of potential adaptive alleles were identified by serial transfers in flasks, large genomic re-arrangements (two columns on the far right) were seen only, and consistently, in the microdroplet evolved populations. A comprehensive list of mutations identified in all populations can be found in Datasets S1-S6.

FIG. 19. Heat map shows fraction of end point isolates carrying a specific mutant allele implicated in DOX resistance from FIG. 18. D1-D5 are microdroplet evolved populations described in Table 1. Data was collected from 3 end point isolates obtained from each replicate population within each experimental evolution condition. Table 3 shows the DOX MICs for each isolate. *Amplification of marA is the result of a 52 kb amplification event. A comprehensive list of all mutations identified in end point isolates can be found in Dataset S8.

FIGS. 20A-F. 10-fold increase in coverage for a 52 kb region in the E. coli BW25113 genome between position 1,585,022 and 1,641,281. FIGS. 20A-B show the read coverage depth in this region. (FIG. 20C) The ends of this region in the reference genome are shown in yellow and green arrows. Based on the read alignment of the evolved population against the reference genome, there is evidence of a new junction, as shown in FIG. 20D. Based on this new junction call, there are two possible genomic re-arrangements: Circularization of this 52 kb region (FIG. 20E) or a tandem amplification event (FIG. 20F) introducing approximately 9 additional copies of this 52 kb region downstream of position 1,641,281. This 52 kb region encodes marR and marAB.

FIG. 21. Area plot showing rise and fall of selected adaptive mutations implicated in DOX resistance during the course of evolution of one population in microdroplets at λ=1. The frequency of the mutation within the population is represented on the Y-axis and the X-axis at the bottom denotes the day of evolution. The X-axis at the top indicates the DOX concentration experienced by the population on a given day. Also shown on this plot are the days when large genomic amplification events were observed in this population (indicated by pink lines). The pink lines correspond to the days on the X-axis on which the amplification was detected but do not correspond to the frequency on the Y-axis.

FIGS. 22A-C. Histograms. (FIG. 22A) Histograms of measured microdroplet diameters with the Gaussian fittings for FIG. 22A 218±6.1 μm microdroplets (Dispersed (W) and continuous (O) phases flow rates: 200 and 200 μl/h), 228±8.6 μm microdroplets (W: 360 and O: 200 μl/h), 260±4.9 μm microdroplets (W: 360 and O: 300 μl/h), (FIG. 22B) 234±3.8 μm microdroplets (W: 1800 and O: 1800 μl/h), and (FIG. 22C) 247±3.5 μm microdroplets (W: 6000 and O: 6000 μl/h).

FIGS. 23A-D. A droplet generator built for λ<1 droplets-based experimental evolution experiments. (FIG. 23A) Schematic of the design of a microfluidic droplet generator. Microscopy images of (FIG. 23B) flow-focusing geometry and (FIG. 23C) observation chamber of a droplet generator with a 60 μm straight downstream channel and 30 μm of the orifice. (FIG. 23D) Histograms of measured microdroplet diameters for 93±2.3 μm microdroplets (W: 1800 and O: 6000 μl/h).

FIG. 24A-B. Serial transfer. (FIG. 24A) Schematic diagram of the serial transfer protocol used for the passage of E. coli to DOX in flask/tubes and (FIG. 24B) selection strengths determined from serial transfer experimental evolution studies by adjusting the gradient of increase in daily DOX concentration.

FIG. 25. DOX concentration gradients and daily population sizes of three replicate populations from microdroplet evolution condition D5.

FIGS. 26A-D. OD₆₀₀ of wild type E. coli (FIGS. 26A-B) and 3-1 end-point isolate (FIGS. 26C-D) from D1 exposed to different DOX concentrations (0, 0.5, 8, and 16 μg/ml). In FIGS. 26A and 26C show growth in a well-mixed flask and in FIGS. 26B and 26D in 247 μm droplets over 24 hours.

FIG. 27. Poisson distribution P(X=x)=λ^{x}e^{-λ}/x!, where P is the probability of finding x cells per droplet and λ is the mean number of cells in the volume of each droplet.

FIGS. 28A-B. Growth characteristics of (FIG. 28A) 1-1 and (FIG. 28B) 2-2 endpoint isolates from D1 exposed to DOX concentrations ranging from 0 to 12 μg/ml. Error bars represent the standard deviation of the results from three biological replicates.

### DESCRIPTION OF ILLUSTRATIVE EMBODIMENTS

As stated above, multi-drug resistant bacteria, along with new and highly dangerous bacterial species, continue to emerge. Thus, there is an increasingly urgent need to identify new, more potent and more specific antibiotics for the treatment of such diseases.

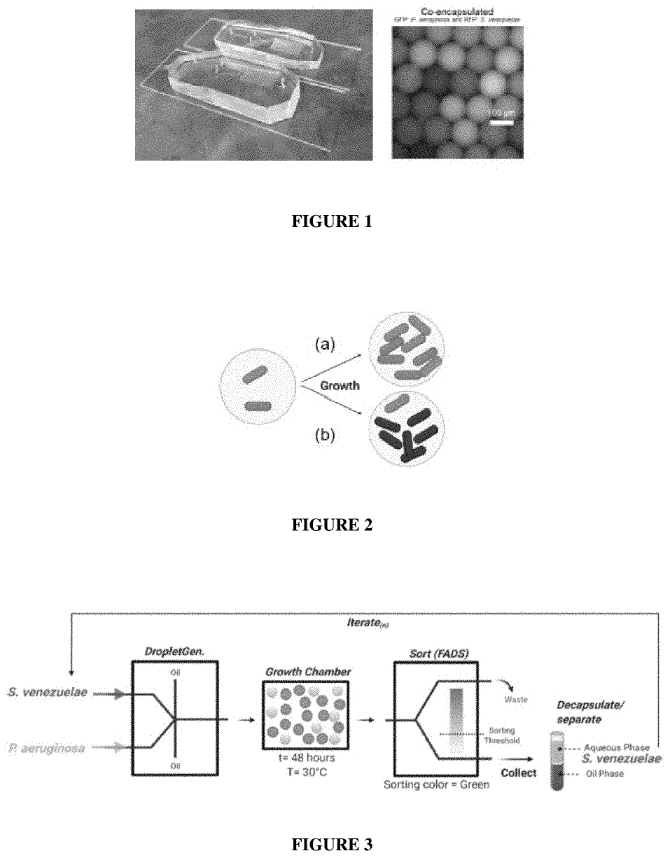

Here, the inventors report the fabrication of improved microfluidic chips for high throughput co-encapsulation of Streptomyces spp. and ESKAPE strains. They employ Fluorescence Activated Droplet Sorting or FADS chips to assemble a technology platform capable of identifying new anti-bacterial agent and have thus, validated a technique they refer to as “MASEDD” for Microfluidic Assembly of Synthetic Ecologies for Drug Discover. The inventors constructed strains suitable for testing MASEDD including a stable chromosomally integrated Green Fluorescent Protein (GFP) expressing ESKAPE pathogen P. aeruginosa PAO1 (P. aeruginosa^{GFP}) for a Prey test strain and S. venezuelae ATCC10712 expressing Red Fluorescent Protein (RFP) (S. venezuelae^{RFP}) as a Predator test strain. They also developed the techniques required for the encapsulation of Streptomyces strains including several wild strains that form large mycelial growth mats that can produce severe device fouling (FIG. 1). In addition, they developed mathematical models to better understand which features of the MASEDD protocol could be made more efficient by tuning parameters such as the number of microdroplets per cycle, microdroplet size, starting Predator:Prey ratios, cell growth rates and other adjustable parameters.

MASEDD allows for the development of highly specific as well as broad-spectrum antimicrobials. It also simultaneously selects for the producer strain that could be used to make the antimicrobial. MASEDD offers an opportunity to address the gaps in antibiotic development pathway in an inexpensive, fast and scalable way. To date, the majority of antibiotics have originated from microbes as natural products. The soil bacteria Streptomyces spp. are a powerful example, having provided about two-thirds of all antibiotics⁸⁻¹⁰. Despite the remarkable contribution of Streptomyces to human health, it is clear that there are still many unknown biochemical pathways that could be exploited to produce new antibiotics if a reliable method by which to drive the exploration of the vast secondary metabolic capabilities of Streptomyces or other organisms existed¹¹⁻¹². MASEDD provides just such a tool and therefore has the potential to dramatically improve future drug discovery.

These and other aspects of the disclosure are described in detail below.

### I. General Assay Design and Features

FIG. 3 shows a schematic for the MASEDD process of selection and enrichment over time (iterations) of Streptomyces variants (Predator) with the ability to inhibit the growth of P. aeruginosa PAO1 (Prey). The Prey strain constitutively expresses Green Fluorescent Protein (GFP) (P. aeruginosa^{GFP}) and model Predator S. venezuelae expresses Red Fluorescent Protein (S. venezuelae^{RFP}). It is not necessary to label the Predator for MASEDD but was useful for initial studies. These are examplary as, in practice, any strain that can be cultured could be a potential Predator strain for use in MASEDD and any bacterial pathogen could be the Prey.

Iterative cycles of evolution to identify and improve candidate Predator strains is important for MASEDD. Starting on the left of FIG. 3, the inventors co-encapsulate mutagenized S. venezuelae^{RFP} with P. aeruginosa^{GFP} into microdroplets (DropletGen). The co-encapsulated micro-populations are allowed to compete and grow until they exhaust the available resources (Growth Chamber). The vast majority of the microdroplets will be green as wild-type S. venezuelae^{RFP} does not inhibit P. aeruginosa^{GFP}. In most cases the cells just compete for food passively as in FIG. 1. After mutagenesis, some Streptomyces mutants may have a weak ability to inhibit P. aeruginosa^{GFP} and those microdroplets will have less green fluorescence as the amount of P. aeruginosa^{GFP} is reduced. They use a second microfluidic device (Fluorescence Activated Droplet Sorting or FADS) to sort the microdroplets on the basis of fluorescence intensity. The inventors keep the microdroplets that have lower green fluorescence as that is consistent with the scenario where a S. venezuelae^{RFP} variant has at least partially inhibited P. aeruginosa^{GFP}. After sorting for the microdroplets with reduced green fluorescence, the microdroplets are broken open by decapsulation. As described previously, Streptomyces grow as larger aggregates and the inventors use a combination of low-speed centrifugation and filtration to reduce the number of Prey cells entering the next cycle. The next cycle is begun by co-encapsulating S. venezuelae^{RFP} with fresh Prey cells. Fresh Prey cells are used in each cycle to reduce the counter-evolution of resistance or accumulation of P. aeruginosa^{GFP} variants with reduced fluorescence that would generate false positives. Over time and with each iteration, the inventors select and enrich for genotypes that have an ability to inhibit P. aeruginosa^{GFP}. At the end of MASEDD, they perform genomic sequencing of promising end-point isolates and characterize the mechanism of inhibition to produce a ranked list of candidate strains for further study. Importantly, each of the successful end-point strains is a producer of an antimicrobial lead molecule and could be used to scale up lead production.

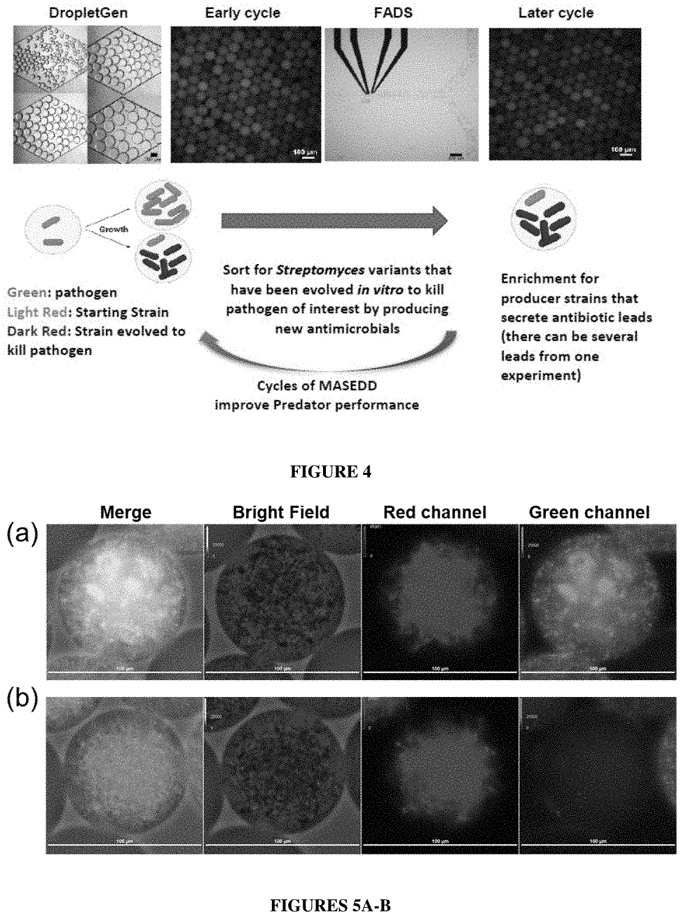

Spatial segregation of cells within microenvironments is important for MASEDD. In a typical batch experiment such as experimental evolution in flasks, the Predator-Prey approach would not work. A single new Predator that begins producing an antimicrobial will not increase in number relative to the general batch population (10⁷-10⁹ cells) because: a) the entire population benefits from the new molecule and are essentially ‘cheaters’; b) the bulk media is well mixed meaning that the small amount of antimicrobial produced by a Predator is diluted to the entire volume of the batch; and c) the new Predator may have a slower growth rate than the ancestor genotype as production of the molecules may be costly thereby putting it on a road to extinction in a batch format^{13, 14}. MASEDD takes advantage of spatial segregation provided by the microdroplets to give each cell safe harbor to grow without cheaters capitalizing on their innovation (FIGS. 4 and 5). Just 2 ml of a 70 μm microdroplet emulsion is equivalent to 10 million spatially segregated experimental chambers. To manually screen the population from a single round of MASEDD (˜10⁹ cells) would require >1 million agar plates per day.

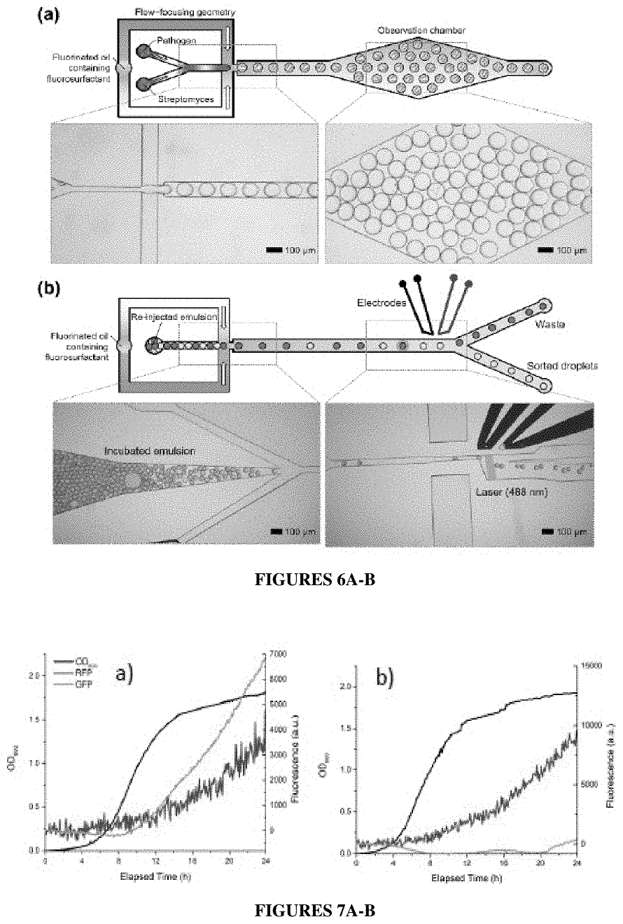

There are two core microfluidic devices important for MASEDD. FIGS. 6A-B provide examples of the microfluidic devices (DropletGen and FADS) and FIG. 4 provides examples of the microdroplet populations at early and later cycles. In FIG. 4, P. aeruginosa^{GFP} and S. venezuelae^{RFP} are labeled with GFP and RFP respectively to make the interpretation of the outcomes easier to visualize. The desired population should be orange-red indicating an ability to inhibit P. aeruginosa^{GFP}.

### II. Prey and Predator Strains

As discussed above, the present disclosure employs a variety of technologies to create microenvironments where pathogenic bacteria can be kept in close proximity to other microbes capable of producing antibiotics. The following discussion focuses on the nature of the prey and predator strains.

A. Prey

In theory, a prey strain according to the present disclosure can be any pathogenic bacteria. This includes both Gram-positive and Gram-negative pathogens as well as drug and multi-drug resistant pathogens. In particular, the pathogen may be an “ESKAPE” pathogen.

ESKAPE is an acronym comprising the scientific names of six highly virulent and antibiotic resistant bacterial pathogens including Enterococcus faecium, Staphylococcus aureus, Klebsiella pneumoniae, Acinetobacter baumannii, Pseudomonas aeruginosa, and Enterobacter spp. This group of Gram-positive and Gram-negative bacteria can evade or ‘escape’ commonly used antibiotics due to their increasing multi-drug resistance (MDR). As a result, throughout the world, they are the major cause of life-threatening nosocomial or hospital-acquired infections in immunocompromised and critically ill patients who are most at risk. P. aeruginosa and S. aureus are some of the most ubiquitous pathogens in biofilms found in healthcare. P. aeruginosa is a Gram-negative, rod-shaped bacterium, commonly found in the gut flora, soil, and water that can be spread directly or indirectly to patients in healthcare settings. The pathogen can also be spread in other locations through contamination, including surfaces, equipment, and hands. The opportunistic pathogen can cause hospitalized patients to have infections in the lungs (as pneumonia), blood, urinary tract, and in other body regions after surgery. S. aureus is a Gram-positive, cocci-shaped bacterium, residing in the environment and on the skin and nose of many healthy individuals. The bacterium can cause skin and bone infections, pneumonia, and other types of potentially serious infections if it enters the body. S. aureus has also gained resistance to many antibiotic treatments, making healing difficult. Because of natural and unnatural selective pressures and factors, antibiotic resistance in bacteria usually emerges through genetic mutation or acquired antibiotic-resistant genes (ARGs) through horizontal gene transfer—a genetic exchange process by which antibiotic resistance can spread.

One of the main reasons for the rise in the selection for antibiotic resistance (ABR) and MDR which led to the emergence of the ESKAPE bacteria is from the rash overuse of antibiotics not only in healthcare, but also in the animal, and agricultural sector. Other key factors include misuse and inadequate adherence to treatment guidelines. Due to these factors, fewer and fewer antibiotic treatments are effective in eradicating ABR and MDR bacterial infections, while at the same time there are now no new antibiotics being created due to lack of funding. These ESKAPE pathogens, along with other antibiotic-resistant bacteria, are an interweaved global health threat and are being addressed from a more holistic and One Health perspective.

Enterococcus faecium. Enterococcus faecium is a Gram-positive spherically-shaped (coccus) bacteria that tends to occur in pairs or chains, most commonly involved in healthcare associated infection (HAI) in immunocompromised patients. It often exhibits a resistance to β-lactam antibiotics including penicillin and other last resort antibiotics. There has also been a rise in vancomycin resistant enterococci (VRE) strains, including an increase in E. faecium resistance to vancomycin, particularly vancomycin-A. These vancomycin-resistant strains display a profound ability to develop and share their resistance through horizontal gene transfer, as well as code for virulence factors that control phenotypes. These virulence phenotypes range from thicker biofilms to allowing them to grow in a variety of environments including medical devices such as urinary catheters and prosthetic heart valves within the body. The thicker biofilms act as a “mechanical and biochemical shield” that protects the bacteria from the antibiotics and are the most effective protective mechanism that bacteria have against treatment.

Staphylococcus aureus. Staphylococcus aureus is a Gram-positive spherically-shaped (coccus) bacteria that is commonly found as a part of the human skin microbiota and is typically not harmful in humans with non-compromise immune systems in these environments. However, S. aureus has the ability to cause infections when it enters parts of the body that it does not typically inhabit, such as wounds. Similar to E. faecium, S. aureus can also cause infections on implanted medical devices and form biofilms that make treatment with antibiotics more difficult Additionally, approximately 25% of S. aureus strains secrete the TSST-1 exotoxin responsible for causing toxic shock syndrome. Methicillin-resistant S. aureus, or MRSA, includes strains distinct from other strains of S. aureus in the fact that they have developed resistance to β-lactam antibiotics. Some also express an exotoxin that has been known to cause “necrotic hemorrhagic pneumonia” in those who suffer from infection. Vancomycin and similar antibiotics are typically the first choices for treatment of MRSA infections, however from this vancomycin-resistant S. aureus, or VRSA (VISA for those with intermediate resistance) strains have emerged.

Klebsiella pneumoniae. Klebsiella pneumoniae is a Gram-negative rod-shaped (bacillus) bacteria that is particularly adept to accepting resistance genes by horizontal gene transfer. It is commonly also resistant to phagocyte treatment due to its thick biofilm with strong adhesion to neighboring cells. Certain strains have also developed β-lactamases that allow them to be resistant to many of the commonly used antibiotics, including carbapenems, which has led to the creation of carbapenem-resistant K. pneumoniae (CRKP), for which there are very few antibiotics in development that can treat infection.

Acinetobacter baumannii. Acinetobacter baumannii is most common in hospitals, which has allowed for the development of resistance to all known antimicrobials. The Gram-negative short-rod-shaped (coccobacillus) A. baumannii thrives in a number of unaccommodating environments due to its tolerance to a variety of temperatures, pHs, nutrient levels, as well as dry environments. The Gram-negative aspects of the membrane surface of A. baumannii, including the efflux pump and outer membrane, affords it a wider range of antibiotic resistance. Additionally, some problematic A. baumannii strains are able to acquire families of efflux pumps from other species, and commonly develop new β-lactamases to improve β-lactam resistance.

Pseudomonas aeruginosa. The Gram-negative, rod-shaped (bacillus) bacteria Pseudomonas aeurginosa is ubiquitous hydrocarbon degrader that is able to survive in extreme environments as well as in soil and many more common environments. Because of this versatility, it survives quite well in the lungs of patients suffering from late-stage cystic fibrosis (CF). It also benefits from the same previously mentioned Gram-negative resistance factors as A. baumannii. Mutants of P. aeurginosa with upregulated efflux pumps also exist that make finding an effective antibiotic or detergent incredibly difficult. There are also some multi-drug resistant (MDR) strains of P. aeruginosa that express β-lactamases as well as upregulated efflux pumps which can make treatment particularly difficult.

Enterobacter spp. Enterobacter encompasses a family of Gram-negative, rod-shaped (bacillus) species of bacteria. Some strains cause urinary tract (UTI) and blood infections and are resistant to multiple drug therapies, which therefore puts the human population in critical need for the development of novel and effective antibiotic treatments. Colistin and tigecycline are two of the only antibiotics currently used for treatment, and there are seemingly no other viable antibiotics in development. In some Enterobacter species, a 5-300-fold increase in minimum inhibitory concentration was observed when exposed to several gradually increasing concentrations of benzalkonium chloride (BAC). Other Gram-negative bacteria (including Enterobacter, but also Acinetobacter, Pseudomonas, Klebsiella species, and more) also displayed a similar ability to adapt to the disinfectant BAC.

B. Predator

Predator strain according to the present disclosure can be any microbe—bacteria or fungi—that is capable of producing an antibiotic substance. The present inventors have employed Streptomyces venezuelae as their predator strain but this is only exemplary and not limiting. Other examples of predator strains include, but are not limited to wild or cultured isolates belonging to the genus Streptomyces, Bacillus, Nocardia and fungi such as Penicillium.

C. Mutagenesis

In order to enhance the ability to identify new and/or improved antibiotics, the predator strain may be subjected to mutagenesis. Mutagenesis is common and long-practiced method by which mutations are induced at a higher than natural rate by subjecting organisms to mutagenic influences, such as drugs or radiation. Such methods are generally considered “random” in that they are not targeted to particular genes or pathways. Other “tailored” mutagenic methods are called site-directed methods.

In random methods, cells or organisms are exposed to mutagens such as UV radiation or mutagenic chemicals, and mutants with desired characteristics are then selected. Alkylating agents such as N-ethyl-N-nitrosourea (ENU) have been used to generate mutant mice. Ethyl methanesulfonate (EMS) is another mutagen. The present inventors employed, for these studies, N-methyl-N′-nitro-N-nitrosoguanidine (NTG).

D. Bacterial Labeling

In some embodiments, it will be useful to identify, quantify and sort microbes—either or both of predator and prey strains. Thus, engineering the microbes to produce detectable signals is specifically contemplated. Using standard technologies, microbes can be engineered to produce fluorescent, chemiluminescent, gas and colorimetric labels. Commonly used examples fluorescent proteins include GFP, EGFP, Emerald, Superfolder GFP, Azami Green, mWasabi, TagGFP, TuroGFP, AcGFP, ZsGreen, T-Sapphire, EBFP, EBFP2, Azurite, mTabBFP, ECFP, mECFP, Cerluean, mTurqoise, CyPet, AmCyanl, Midori-Ishi Cyan, TagCFP, mTFP1, WYFP, Topaz, Venus, mCitrine, YPet, TagYFP, PhiYFP, ZsYellow1, mBanana, Kusabira Orange, Kusabira Orange2, mOrange, mOrange2, dTomato, dTomato-Tandem, TagRFP, TagRFP-T, DsRed, DsRed2, DsRed-Express, DeRed-Monomer, mTangerine, mRuby, mApple, mStrawberry, AsRed2, mRFP1, JRed, mCherry, McRed1, mRaspberry, dKeima-Tandem, HcRed-Tandem, mPlus and AQ143. Currently available genetic tools including, but not limited to, homologous recombination, transposon mediated insertion and CRISRP mediated mutagenesis will be used to chromosomally integrate a copy of the fluorescence gene under the control of a constitutive promoter into either or both of predator and prey strains.

### III. Microencapsulation

Microencapsulation is a process in which tiny particles or droplets are surrounded by a coating to produce small, isolated environments with useful properties. In general, it is used to incorporate food ingredients, enzymes, cells or other materials on a micrometric scale. Microencapsulation can also be used to enclose solids, liquids, or gases inside a micrometric wall made of hard or soft soluble film, in order to reduce dosing frequency and prevent the degradation of pharmaceuticals.

In its simplest form, a microcapsule is a small sphere enclosing some material. The inventors have employed a microfluidic chip for generating emulsions of microdroplets of varying user designed sizes and encapsulating the two strains (DropletGen) (FIGS. 6A-B). The inventors can make microdroplets of varying sizes (50-250 μm) that have the capacity to hold from 1 to ˜1600 cells. They typically introduce a small number of cells of each strain as the founding population in each microdroplet. Depending on the size of the microdroplet, growth rate, resources, and cell size, each microdroplet typically supports from 4-8 doublings which is comparable to the number of doublings that occur in a typical experimental evolution study using flasks. One milliliter of emulsion (microdroplet size 90 μm and volume ˜380 μl) contains about ˜2.6 million droplets and thus each milliliter of emulsion can be thought of as having ˜2.6 million tiny bioreactors as spatially independent experimental evolution studies. The inventors' latest generation of DropletGens can produce 1 ml of emulsion of 90 μm microdroplets in 30 minutes. The flow-focusing geometry of a microfluidic chip for generating emulsions of microdroplets is shown (FIG. 6A). The growth media containing two distinct strains are introduced into the junction in the middle microchannel while the fluorinated oil is injected on both sides. A biocompatible non-ionic fluorosurfactant (for example: Pico-Surf™, Sphere Fluidics) is used to prevent droplet coalescence. After the microdroplets containing cells are produced, they are placed into a GrowthChamber. Emulsions cannot be shaken without risking coalescence of the microdroplets and therefore the GrowthChamber must provide constant conditions without dehydration and allow for surface area sufficient for good aeration. While vigorous shaking is generally not well tolerated by emulsions, nutation and increasing head space volume within the tubes containing the emulsions is used to increase aeration.

### IV. Microfluidics

Microfluidics refers to the behavior, precise control, and manipulation of fluids that are geometrically constrained to a small scale (typically sub-millimeter) at which surface forces dominate volumetric forces. It is a multidisciplinary field that involves engineering, physics, chemistry, biochemistry, nanotechnology, and biotechnology. It has practical applications in the design of systems that process low volumes of fluids to achieve multiplexing, automation, and high-throughput screening. Microfluidics emerged in the beginning of the 1980s and is used in the development of inkjet printheads, DNA chips, lab-on-a-chip technology, micro-propulsion, and micro-thermal technologies.

Typically, micro means one of the following features:
Small volumes (μL, nL, pL, fL)
Small size
Low energy consumption
Microdomain effects
   Typically, microfluidic systems transport, mix, separate, or otherwise process fluids. Various applications rely on passive fluid control using capillary forces, in the form of capillary flow modifying elements, akin to flow resistors and flow accelerators. In some applications, external actuation means are additionally used for a directed transport of the media. Examples are rotary drives applying centrifugal forces for the fluid transport on the passive chips. Active microfluidics refers to the defined manipulation of the working fluid by active (micro) components such as micropumps or microvalves. Micropumps supply fluids in a continuous manner or are used for dosing. Microvalves determine the flow direction or the Zode of movement of pumped liquids. Often, processes normally carried out in a lab are miniaturised on a single chip, which enhances efficiency and mobility, and reduces sample and reagent volumes.

The behavior of fluids at the microscale can differ from “macrofluidic” behavior in that factors such as surface tension, energy dissipation, and fluidic resistance start to dominate the system. Microfluidics studies how these behaviors change, and how they can be worked around, or exploited for new uses.

At small scales (channel size of around 100 nanometers to 500 micrometers) some interesting and sometimes unintuitive properties appear. In particular, the Reynolds number (which compares the effect of the momentum of a fluid to the effect of viscosity) can become very low. A key consequence is co-flowing fluids do not necessarily mix in the traditional sense, as flow becomes laminar rather than turbulent; molecular transport between them must often be through diffusion.

High specificity of chemical and physical properties (concentration, pH, temperature, shear force, etc.) can also be ensured resulting in more uniform reaction conditions and higher-grade products in single and multi-step reactions.

Microfluidic flows need only be constrained by geometrical length scale—the modalities and methods used to achieve such a geometrical constraint are highly dependent on the targeted application. Traditionally, microfluidic flows have been generated inside closed channels with the channel cross section being in the order of 10 μm×10 μm. Each of these methods has its own associated techniques to maintain robust fluid flow which have matured over several years.

Microfluidic flow focusing droplet generation. Using microfluidic devices, droplets are generated when two immiscible liquid phases are mixed together in a controlled manner. This process can be accomplished either by active on-demand droplet production methods that use external forces (i.e., electrical, centrifugal, optical, and magnetic fields) to split droplets or passive droplet production methods for continuous droplet production using pressure-driven flow (flow focusing). Flow focusing is one of the passive droplet production methods and employs straightforward hydrodynamic principles. In comparison with other active droplet production techniques such as electrospray, one of the advantages of passive droplet production methods is that no external energy input from external elements is required, except for traditional liquid pumping.

In a flow-focusing geometry, when the dispersed phase is hydrodynamically focused by a continuous phase on both sides, the dispersed phase creates a thin neck near the junction where both fluids intersect. Once a neck reaches the critical thickness, this neck is pinched-off, generating spherical droplets at very high rates (kHz). Depending on the force balance (i.e., capillary, viscous, and inertial forces) in the microchannel, microdroplets can be produced in different flow regimes i.e., dripping, squeezing, and jetting regimes. The Reynolds number which is the ratio of inertial force to viscous force typically ranges between 10⁻⁶ and 10. Since the viscous force dominates over inertial force, flow in the microchannel is typically laminar flow. Generated droplets in the continuous phase flow in parallel layers downstream as a laminar flow without mixing. One of the non-dimensional numbers, the capillary number, which is the ratio of viscous force to interfacial tension is typically in the range of 10−3 to 10. Squeezing regimes for smaller droplets with higher throughput leading to higher manufacturing efficiency occurs at low capillary numbers, such as Ca<10⁻². The microdroplet size can be modified by varying intrinsic properties of the dispersed and continuous phases (i.e., flow rate, interfacial tension, and viscosity).

### V. Fluorescence-Activated Droplet Sorting

As discussed above, droplet-based microfluidics manipulate discrete volumes of fluids in immiscible phases with low Reynolds number and laminar flow regimes. Microdroplets offer the feasibility of handling miniature volumes (μl to fl) of fluids conveniently, provide better mixing, encapsulation, sorting, sensing and are suitable for high throughput experiments. Two immiscible phases used for the droplet-based systems are referred to as the continuous phase (medium in which droplets flow) and dispersed phase (the droplet phase).

One advantage to droplet-based microfluidics is droplet sorting, allowing for discrimination based on factors ranging from droplet size to chemicals labeled with fluorescent tags within the droplet, stemming off of the work done to sort cells in Flow Cytometry. Within the realm of droplet sorting there are two main types, bulk sorting, which uses either active or passive methods, and precise sorting, which relies mainly on active methods. Bulk sorting is applied to samples with a large number of droplets (>2000 s⁻¹) that can be sorted based on intrinsic properties of the droplets (such as viscosity, density, etc.) without checking each droplet. Precise sorting, on the other hand, aims to separate droplets that meet certain criteria that is checked on each droplet.

Passive sorting is done through control of the microfluidic channel design, allowing for discrimination based on droplet size. Size sorting relies on the bifurcating junctions in the channel to divert the flow, which causes droplets to sort based on how they interact with the cross section of that flow, the shear rate, which relates directly to their size. Other passive methods include inertia and microfiltration, each having to do with the physical properties, such as inertia, and density, of the droplet. Active sorting uses additional devices attached to the microfluidic device to alter the path of a droplet during flow by controlling some aspect, including thermal, magnetic, pneumatic, acoustic, hydrodynamic and electric control. These controls are utilized to sort the droplets in response to some signal detection from the droplets such as fluorescence intensity.

Precise sorting methods utilize these active sorting methods by first making a decision (e.g., fluorescence signal) about the droplets then altering their flow with one of the aforementioned methods. A technique called Fluorescent Activated Droplet Sorting (FADS) has been developed which utilizes electric field-induced active sorting with fluorescent detection to sort up to 2000 droplets per second. The method relies on, but is not limited to, enzymatic activity of compartmentalized target cells to activate a fluorogenic substrate within the droplet. When a fluorescing droplet is detected, two electrodes are switched on applying a field to the droplet, which shifts its course into the selection channel, while non-fluorescing droplets flow through the main channel to waste. Alternatively, non-fluorescing or droplets with lower fluorescence intensity can be collected depending on whether the Predator or Prey is labelled. Other methods utilize different selection criteria, such as absorbance of droplet, number of encapsulated particles, or image recognition of cell shapes. Sorting can be done to improve encapsulation purity, an important factor for collecting sample for further experiments.

An important factor for MASEDD device is a Fluorescence Activated Droplet Sorting (FADS) chip (FIG. 6B). Whenever possible, strains are fluorescently labeled by the production of Green Fluorescent Protein (GFP), Red Fluorescent Protein mCherry (RFP) or as in Aim 2, mGreenLantern. Fluorescence allows the inventors to sort droplets or populations by color using FADS. FADS chips are typically mounted on the stage of an inverted fluorescence microscope (Nikon Ti2E) equipped with a high-speed camera (Phantom V7.2) that allows the inventors to evaluate sorting and chip performance. FADS chips can also be daisy chained to allow for sequential color sorting. After cell growth, the droplets can then be sorted either for the presence of fluorescence (e.g., red or green) or by total fluorescence (e.g., how green is the microdroplet?). Sorting on the basis of the intensity of a color is referred to as the “sorting threshold” (FIG. 3). FIG. 6B shows a typical FADS chip. The sorted droplets are then disrupted to release cells for use in subsequent steps.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—MASEDD Demonstration

The inventors performed 3 rounds of MASEDD using P. aeruginosa^{GFP} as Prey and chemically mutagenized S. venezuelae^{RFP} as Predator. Using the protocol shown in FIGS. 3 and 4, they were able to isolate S. venezuelae^{RFP} variants that strongly inhibit P. aeruginosa^{GFP} (FIGS. 7A-B). To test these candidate strains, 60 individual MASEDD evolved S. venezuelae^{RFP} isolates were co-incubated with fresh P. aeruginosa^{GFP}. After 24 hours of incubation 57 of 60 were able to inhibit P. aeruginosa^{GFP} (FIG. 7B). These results support the ability of MASEDD to enrich for variant Predators that can inhibit Prey cells. If the S. venezuelae^{RFP} is not initially subjected to mutagenesis there is no evidence of a subpopulation that can inhibit P. aeruginosa^{GFP}. The result from the unmutagenized S. venezuelae^{RFP} population is a valuable control as it shows that MASEDD is identifying new variants previously inaccessible using more convention screening methods even in a well characterized model strain.

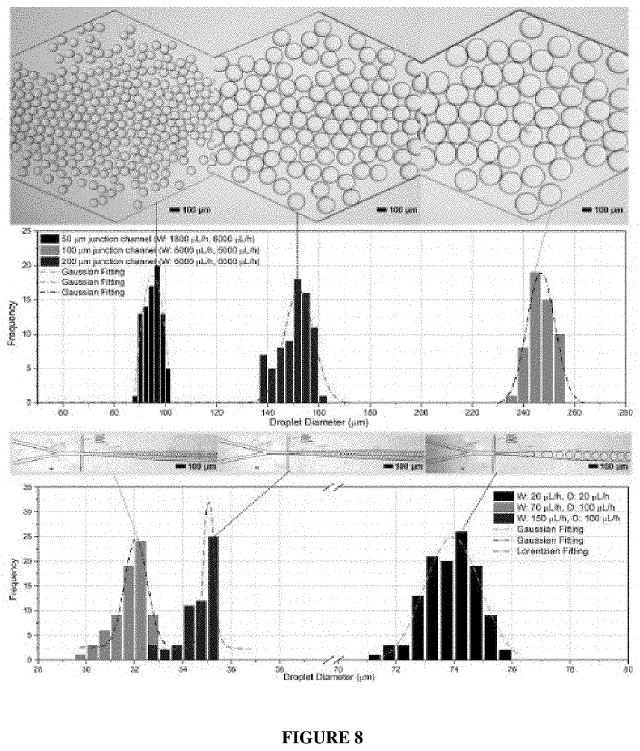

As shown in FIG. 8, the inventors can manufacture microdroplets of high homogeneity over a wide range of sizes (50-250 μm) using current DropletGen devices. The larger the microdroplet the larger the “carrying capacity” for cells. Using E. coli as an example, a 250 μm microdroplet can hold ˜1600 cells while a 90 μm microdroplet can carry ˜86 cells. 90, 150, and 250 μm microdroplets have proven useful in allowing the isolation of potential candidate Predator strains.

The inventors have developed very high throughput DropletGen chips that can produce 2.6 million 90 μm microdroplets in 30 minutes using 1800 μl/h and 6000 μl/h for cell suspension flow rates, and 0.5 million 150 μm microdroplets and 0.1 million 250 μm microdroplets in 10 minutes using 6000 μl/h for cell suspension flow rates, but there are significant advantages to using smaller microdroplets as it would increase the number of candidate Predators they could screen with each cycle. For example, 1 ml of 90 μm microdroplet emulsion has ˜2.6 million microdroplets while a 35 μm microdroplet emulsion has ˜45 million. More microdroplets per ml effectively increases the number of candidates that can be enriched by MASEDD.

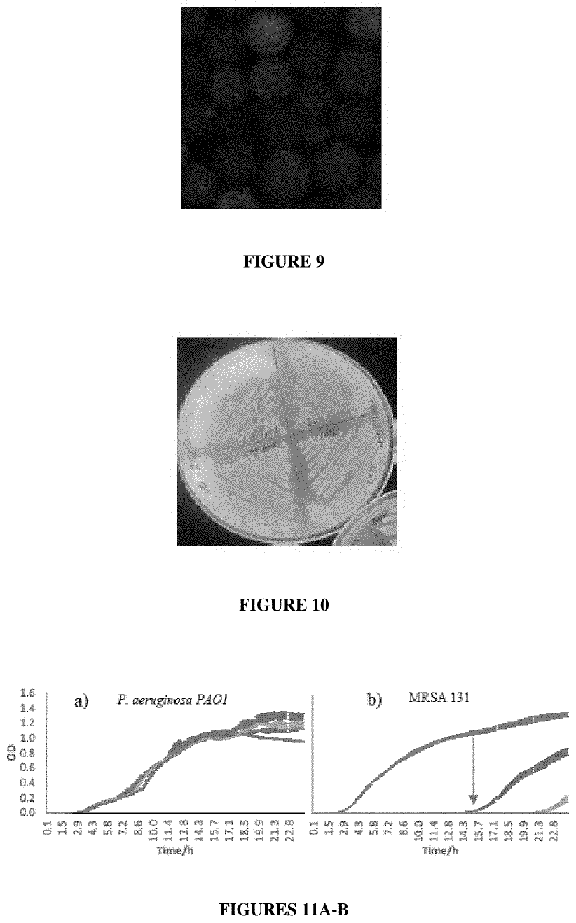

To date, 90 μm DropletGen devices have proven to be suitable for the challenging growth characteristics of Streptomyces strains. Although Streptomyces cells are about the same size as the ESKAPE pathogens, they do not typically grow as single cells but rather as large mycelial mats that easily foul the small channels of microfluidic devices (FIG. 9). The inventors have shown that Streptomyces strains, including wild-type ones, successfully sporulate in microdroplets and by using a combination of larger channels and removal of larger mycelial fragments by filtration, the inventors have been able to passage Streptomyces successfully across the cycles of MASEDD. The size of a microdroplet determines three important parameters: 1) the number of cells that can grow within a microdroplet (carrying capacity) which determines the number of cell divisions per microdroplet; 2) the number of microdroplets per ml which determines the number of mutants that can be screened for activity and 3) the fluorescence of each microdroplet. These parameters trade off against each other and can be optimized for MASEDD efficiency. While successful in their initial studies, the inventors predict that MASEDD efficiency could be improved by using moderately smaller microdroplets to increase the number of candidate Predators in each cycle.

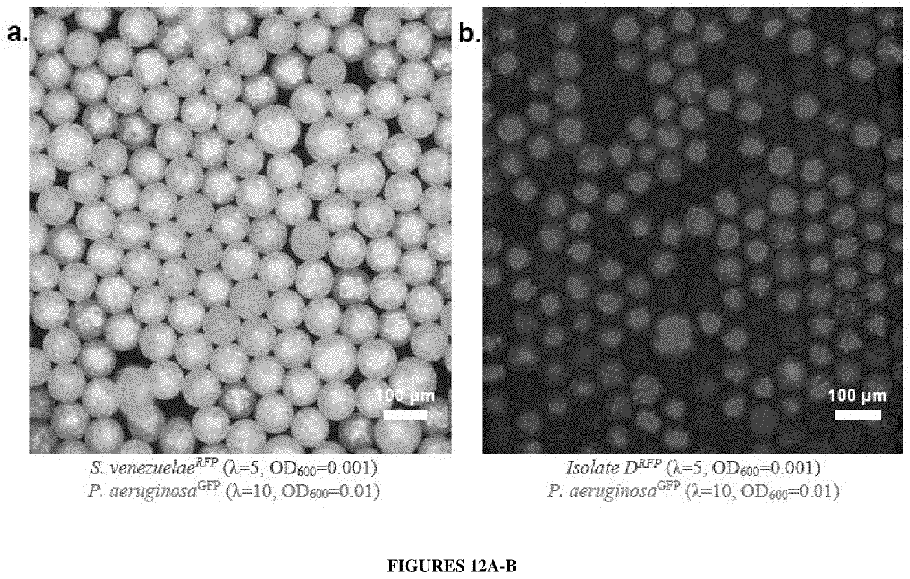

MASEDD isolation of candidate Predator variants. The inventors performed MASEDD using P. aeruginosa^{GFP} as Prey and chemically mutagenized S. venezuelae^{RFP} as Predator. They were able to enrich droplets containing S. venezuelae^{RFP} variants that strongly inhibit P. aeruginosa^{GFP}. Importantly, the ancestor S. venezuelae does not inhibit the growth of Pa^{GFP} in microdroplets. See FIGS. 12A-B. This is not surprising as P. aeruginosa PAO1 is resistant to a wide range of antibiotics including chloramphenicol (MIC=32 μg/ml).

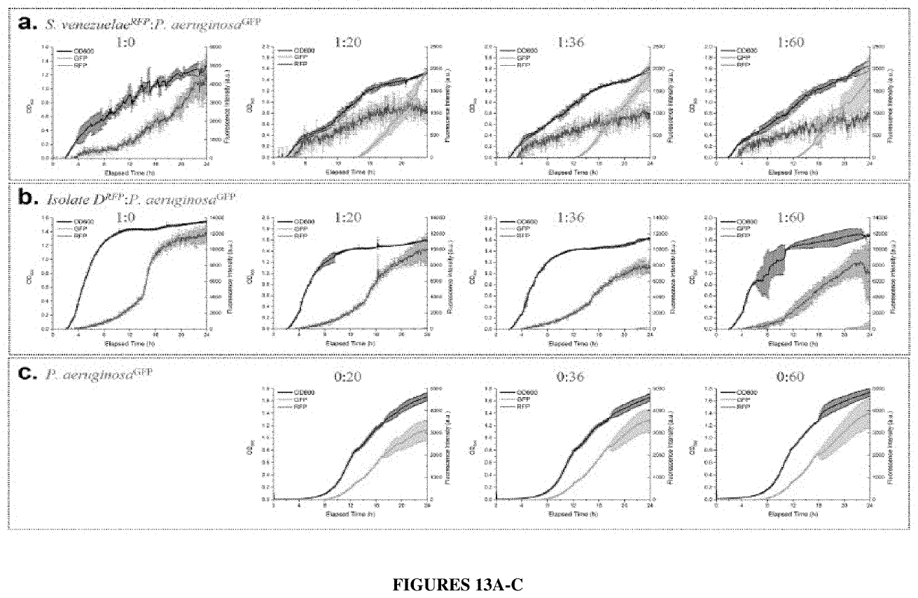

Co-culturing of MASEDD isolates for inhibition activity in a 96-well plate assay. To test candidate strains, 30 individual MASEDD evolved S. venezuelae^{RFP} isolates were co-incubated with fresh P. aeruginosa^{GFP}. Four of 30 isolates were able to inhibit P. aeruginosa^{GFP} in a co-culturing assay. The inventors went on to further characterize the 4 individual sub-strains (A, B, C, D). Results for Isolate D are shown below (FIGS. 13A-C). The ratio of Isolate D (Predator) to Prey was varied from left to right in each panel to assess how strong the inhibition effect was. For example, in panel b there is very little growth P. aeruginosa^{GFP} even at a ratio of 1 Isolate D to 60 P. aeruginosa^{GFP}. By comparison, the ancestor (panel a) showed clear evidence of P. aeruginosa^{GFP} growth at the lowest competition ratio. These preliminary results support the ability of MASEDD to enrich for variant Predators that can inhibit Prey cells.

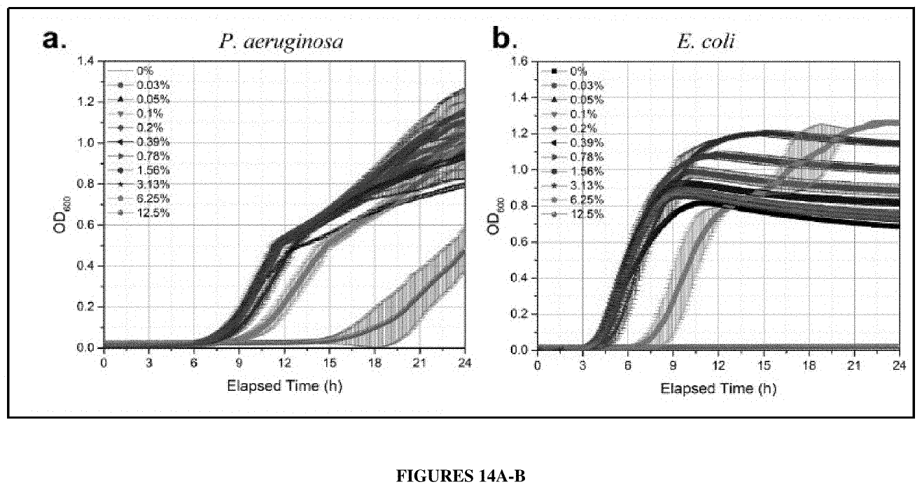

Spent media assays show that the antimicrobial activity of Isolate D is secreted into the media. Spent media was prepared from a monoculture of Isolate D growing in a 1 liter batch format. Cells were removed by centrifugation and then filtration (0.22 μm). Spent media was then lyophilized to dryness and reconstitututed at 20× in water. Spent media inhibited the growth of both P. aeruginosa and E. coli. This data suggested that the inhibition of E. coli was stronger but in both cases was dose dependent. Spent media prepared from the original strain does not have strong activity against either species. This data shows that the antimicrobial activity can be recovered and that the Isolate D makes the molecule(s) even under conventional laboratory growth conditions. See FIGS. 14A-B.

Genomic Analysis of Isolates A-D. The inventors have also performed comparative genomic sequencing and preliminary analysis suggests strains C and D have genetic changes in a putative and uncharacterized biosynthetic pathway that includes enzymes associated with small molecule synthesis including potential antimicrobials.

Based on their preliminary data, the inventors believe the “sweet spot” of optimizing the number of mutant candidate Predator cells and the number of generations of growth that is a function of carrying capacity of the microdroplet is likely <75 μm. The smaller droplets occupy a smaller volume and improve efficiencies at the subsequent sorting steps. For example, a 1 ml emulsion of 35 μm microdroplets has ˜45 million microdroplets compared to the ˜2.6 million in 1 ml of a 90 μm microdroplet emulsion. More droplets would expand the number of variants that could be screened per cycle of MASEDD. A significant challenge in using microdroplets this small is the fouling from Streptomyces strains. The aggregation properties of Streptomyces also preclude use of other technologies such as cell sorting and thus the pressure and fouling properties of devices become more challenging as the inventors move to the smaller channels. The inventors will evaluate whether small molecule modifiers of Streptomyces phenotypes could also be beneficial to reducing clumping and aggregation. Recently, it has been shown that trimethylamine can stimulate Streptomyces sp. exploratory behaviour, by adopting non-branching mycelial structures^{15, 16}. For example, trimethylamine could be evaluated for inclusion in emulsions to reduce mycelia mat formation¹⁶. In addition to S. venezuelae, wild strains S. T4-11 and S. DM5 have also been encapsulated to demonstrate the robustness of MASEDD for non-model organisms.

The inventors already have extensive experience in molecular genetics and characterization of ESKAPE pathogens including vancomycin and daptomycin resistant E. faecalis and E. faecium¹⁷⁻³¹, A. baumannii³² and P. aeruginosa³³. They constructed a P. aeruginosa PAO1 strain containing a chromosomally encoded GFP gene using the mini-Tn7 vector for integration of a single copy gene into a bacterial chromosome³⁴. The same mini-Tn7 vector used to label P. aeruginosa PAO1 can also be used to chromosomally label A. baumannii since it is a broad host range vector and both strains only have a single att7 insertion site. The inventors will genetically manipulate fastidious pathogens to create genomic integrations of the mGreenLantern gene into the strains identified in Table 1 such as methicillin resistant S. aureus, carbapenem resistant K. pneumoniae³⁵ and E. cloacae³⁶. All these methods are based on a double crossover technique mediated by a suicide vector encoding a selection and counter-selection marker. Table 1 shows the list of ESKAPE pathogens the inventors will use to generate a library of fluorescently labelled prey strains.

TABLE 1
ESKAPE pathogens that will be labelled for constitutive
expression of the nextgen fluorescent reporter
mGreenLantern from the genome.

| | Strain | Variant | Primary drug resistance |
|---|---|---|---|
| E | E. faecium | HOU503 2D3-2 | Vancomycin(VAN)/ |
| | | | Daptomycin(DAP) |
| S | S. aureus | MRSA 131 | Methicillin |
| K | K. pneumoniae | ST258 | Carbapenems |
| A | A. baumannii | AB210M adeS^{D167A} | Tigecycline (TIG) |
| P | P. aeruginosa | PAO1 v17 | Colistin (CST) |
| E | Enterobacter | ATCC13047 | Type strain |
| | cloacae | | |

Candidate variants will be screened for antimicrobial activity against the Prey using a simple Conditioned Media Assay. Candidate cell lines are grown to stationary phase in liquid culture and the cells removed subsequently by centrifugation/filtration. The supernatant or “conditioned media” is then added in different amounts to fresh media inoculated with Prey cells and allowed to grow over 24 hours in a 96-well plate where the optical density is measured over time. FIGS. 11A-B show an example of this crude conditioned media assay and shows results from an unknown lead derived from a wild strain of Streptomyces. In this example, the unpurified lead molecule had antimicrobial activity against Gram-positives but no activity against a Gram negative.

### Example 2—Materials and Methods

Microfluidic chips fabrication. The microchannel geometry design provided as Supplementary Data was drawn using computer-aided design (CAD) software (AutoCAD 2018, Autodesk, Inc., Sausalito, CA). Then, the design was printed onto a high-resolution transparency sheet (25,400 dpi, CAD/ART Services Inc., Bandon, OR) to fabricate a patterned photomask. A layer of the negative photoresist (SU-8 2075, Microchem, Newton, MA) was deposited on 4-inch silicon (Si) wafer (UniversityWafer, Inc., Boston, MA) with a spin coater. The spin-coated photoresist on a silicon wafer was exposed to UV light through a high-resolution photomask using a mask aligner (EVG 620, EV Group, Austria). During UV exposure, photomask patterns from the transparency sheet were transferred to the Si wafer. The regions of the photoresist exposed to UV light became insoluble in a developing solution, whereas the unexposed areas were dissolved, finally fabricating a master mold. Polydimethylsiloxane (PDMS) (Sylgard 184, Dow Corning, Midland, MI) resin and its cross-linker were mixed at a ratio of 10:1 for the polymerization of the PDMS. Degassed PDMS prepolymer was poured onto the master mold. After curing in an oven at 80° C. for 1 h, the PDMS layer was peeled off from the master mold. Inlet and outlet holes were made on the PDMS layer using a 0.5 mm diameter biopsy punch (Integra Miltex, Inc., Germany). The PDMS layer stamp and a glass microscope slide (25×75×1.0 mm, Fisher Scientific, Fair Lawn, NJ) were put into a plasma cleaner (Plasmod, March Instruments, Inc., Concord, CA). The hydroxyl (OH) groups were formed onto the surfaces of the PDMS stamp and glass slide by oxygen plasma treatment. The PDMS stamp was covalently bonded to the glass slide by OH groups reacted with the silanes.

Experimental protocol. Five populations of E. coli were adapted to DOX in flasks to identify the selection gradient for adaptation to DOX. On the first day, six different colonies were used to inoculate control and five populations containing fresh media. The growth media for E. coli was Lysogeny broth (LB; 10 g/l tryptone, 5 g/l yeast extract, 5 g/l sodium chloride). The following day, each population was transferred to fresh media containing 1 μg/ml DOX with 100-fold dilutions. The fresh media containing desired DOX concentration was prepared by appropriate dilution of DOX stock solution. The stock solution was prepared by dissolving DOX (Tokyo Chemical Industry Co., LTD, Japan) in water, followed by filter sterilization through a 0.22 μm filter (Millex-GP, Millipore, Bedford, MA). The following day, each population was transferred to three tubes containing 1×, 1.5×, and 2× of the current DOX concentration. Hereafter, each population with the best growth was transferred into three new tubes while the control population was propagated in fresh media until twice the DOX clinical breakpoint (16 μg/ml) was reached. In a similar way, on the first day of serial transfer of E. coli to DOX in microdroplets, four to six different colonies were used to inoculate control and three to five populations containing fresh media. The following day, the growth media containing a starting size of ˜2.6×10⁶ cells/ml was prepared for each population through appropriate dilution of overnight culture. Fluorinated oil (Novec 7500, 3M, St Paul, MN) with 1.5 w/w % surfactants (Pico-Surf 1™, Sphere Fluidics, Cambridge, UK) was the continuous phase while the growth media containing E. coli was the dispersed phase. These prepared continuous and dispersed phases were injected into four to six microfluidic chips using twelve-multi channel syringe pumps (New Era Pump Systems, Farmingdale, NY) with determined flow rates. More than 800 μl droplets for each population were collected in either a 1.5 ml microfuge tube or a 50 ml tube for the incubation at 37° C. After incubation for 24 h, these droplets were chemically broken with a demulsifier (1H, 1H, 2H, 2H-Perfluorooctanol, >97%, Alfa Aesar, Ward Hill, MA). After OD measurement, cells were propagated by dilution in fresh growth media containing higher DOX concentrations until a concentration of 16 μg/ml. Experiment images were captured using a bright-field inverted microscope (Eclipse Ti2, Nikon, Japan) with a mounted high-speed camera (Phantom V7.2).

Whole genome sequencing and analysis. Final day populations for each evolution condition were sequenced along with 2 to 4 end point isolates from each population. Each daily sample from replicate 1 of the droplet evolved population at k=1 was also subjected to WGS. Genomic DNA from all the samples was extracted using Qiagen DNeasy Ultracean Microbial Kit (Cat No./ID: 12224) following manufacturer's instructions. Illumina compatible libraries were prepared using plexWell™ 384 NGS multiplexed library preparation kit from segWell™. Pooled libraries were sent to a commercial facility for whole genome sequencing with at least 100× coverage on the Illumina HiSeq platform to obtain 2×150 bp reads. Short reads from the re-sequenced E. coli BW25113 strain were aligned to the whole genome sequence of BW25113 obtained from NCBI (NCBI Reference Sequence: NZ_CP009273.1) using the software breseq version 0.33.2⁴⁷ and identified differences were applied to the reference sequence using the APPLY function of breseq (gdtools) to obtain a modified reference. This modified sequence was used as the reference sequence for all subsequent comparisons. All populations were run using the polymorphism flag-p with the default 5% cut-off frequency. All end point isolates were run using the default consensus mode. The NOTS cluster run by Rice University's Center for Research Computing (CRC) was used to run these operations (This work was supported in part by the Big-Data Private-Cloud Research Cyberinfrastructure MRI-award funded by NSF under grant CNS-1338099 and by Rice University).

### Example 3—Results

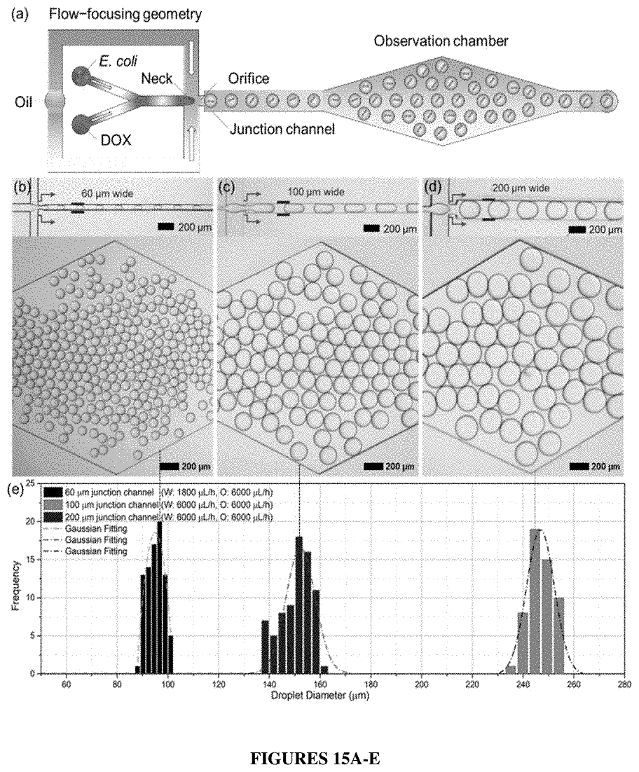

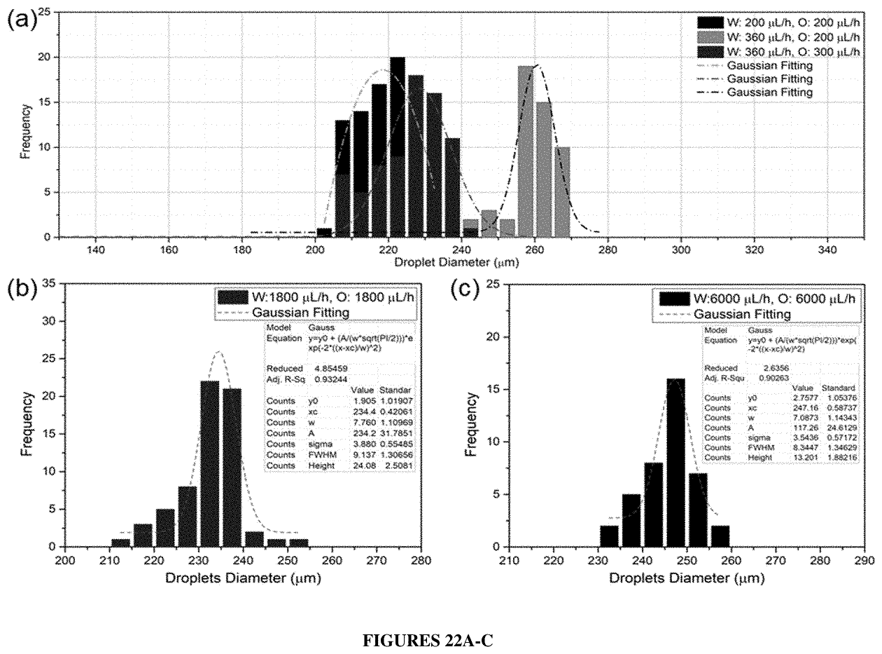

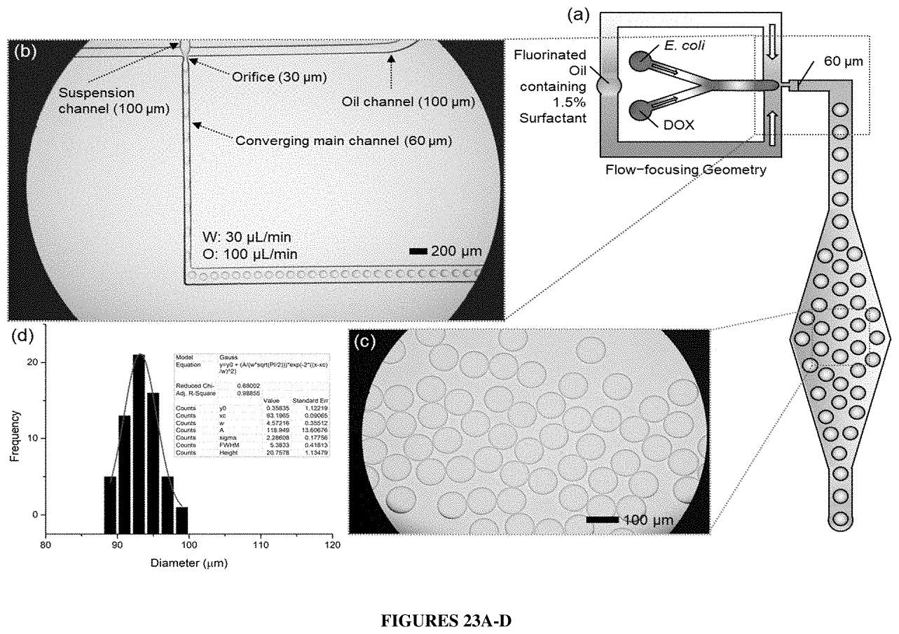

Rapid production of homogenous microdroplets for experimental evolution. In a serial transfer study for experimental evolution in flasks, a starting population of ˜10⁶ cells is often used for each founding population. After growth to stationary phase, the founding population is transferred to a new flask containing an increased concentration of antibiotic. With each successive transfer the antibiotic concentration is increased to select for adaptive genotypes with increased fitness to the selection conditions. The inventors' initial goal was to produce microemulsions that would allow us to compare and contrast the successful evolutionary trajectories identified from microemulsions with those identified from the well-established serial transfer in flask studies. To reproduce conditions comparable to serial transfer experiments in flasks, the inventors needed to rapidly produce a large number of homogeneous microdroplets (>10⁶) with a sufficient carrying capacity for 6-8 doublings, and to provide sufficient numbers of cells after growth for daily metagenomic surveys. As shown in FIGS. 15A-B, the inventors developed and evaluated microfluidic devices that could produce >10⁶ homogeneous microdroplets over a range of sizes from 93-247 μm in less than 35 mins. One of the parameters they wished to vary was the mean starting number of cells per microdroplet (λ) over a range of 1-20 for different studies. For this, the inventors used microdroplets with varying diameters and carrying capacities (the maximum number of cells each microdroplet can hold) so as to achieve comparable number of doublings. Microdroplets of 247±3.5 μm with a carrying capacity ˜1600 E. coli were used for studies with λ=20, while 93±2.3 μm and a carrying capacity of ˜130 E. coli were used for studies using λ≤1 to yield 6-7 doublings in both cases. Microdroplet features such as size and generation frequency are strongly affected by the flow-focusing geometry of the devices the inventors designed for these studies¹⁶⁻¹⁸. As shown in FIGS. 15A-B, the water phase containing cells creates a thin neck near the junction, and this neck is squeezed by the oil phase on both sides. Once the neck reaches the critical thickness, it is pinched off, generating droplets^{19,20}. The microdroplet size is determined by the channel dimensions and the width and depth at the point where the water neck is pinched off²¹. Three designs having junction channel widths of 60, 100, and 200 μm were produced to control microdroplet size. All three designs have an orifice which produces a channel contraction at the junction to increase the shearing force of the continuous oil phase leading to an early breakup point near the orifice^{22,23}. Monodisperse 93±2.3 μm, 153±3.9 μm, and 247±3.5 μm microdroplets were efficiently produced with <3% volume variation (2.5%, 2.6%, and 1.4% respectively). Flow rate studies to rapidly produce and collect the desired number of microdroplets are shown in FIGS. 22A-C and FIGS. 23A-D. Overall, the microdroplet production rates are 4.3×10^{6, 3.2}×10⁶, and 7.6×10⁵/h. Parallelized microfluidic droplet generators were used to achieve production rates that are comparable to that of polydisperse microdroplets (10⁶-10¹⁰/h)¹¹.

Comparison of experimental evolution population dynamics conducted by a serial-transfer methodology to microemulsions suggests microemulsions may provide a stronger selection environment although further experiments are required. Six experimental evolution studies of E. coli BW25113 adaptation to DOX were carried out in microemulsions using varying selection gradients and λ values and compared to serial transfer methods in a well-mixed suspension culture condition (Table 2). DOX is a member of the tetracycline class of antibiotics and binds within the 30S ribosomal subunit²⁴. Like other tetracyclines, DOX is bacteriostatic. Resistance to DOX is typically generated by changes in transporter genes such as the acrAB-tolC family that increase export of the drug or in ribosomal protection genes such as tetM^{25,26}. In rare cases tetracycline resistance can be mediated by members of the TetX oxidoreductase family, but since there is no tetX or tetM allele in E. coli BW25113, the inventors anticipated that resistance would be mediated by transport rather than enzymatic inactivation or ribosomal protection.

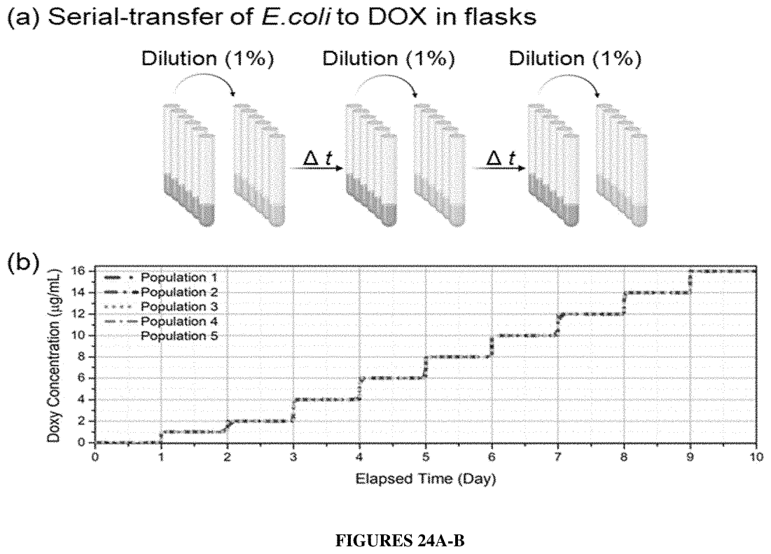

a) Serial-transfer of E. coli to DOX in flasks (F in Table 2). A selection gradient for adaptation to DOX was identified using well-established serial transfer protocols from the inventors' previous studies²⁷⁻²⁹. The antibiotic selection gradient was maintained below the population minimum inhibitory concentration (MIC) and the experiment was terminated when the populations achieved a population MIC of two-times the clinical breakpoint to DOX (FIGS. 24A-B). Five populations of E. coli were established and adapted to DOX over 10 days. A sixth population served as a no-drug control without DOX.

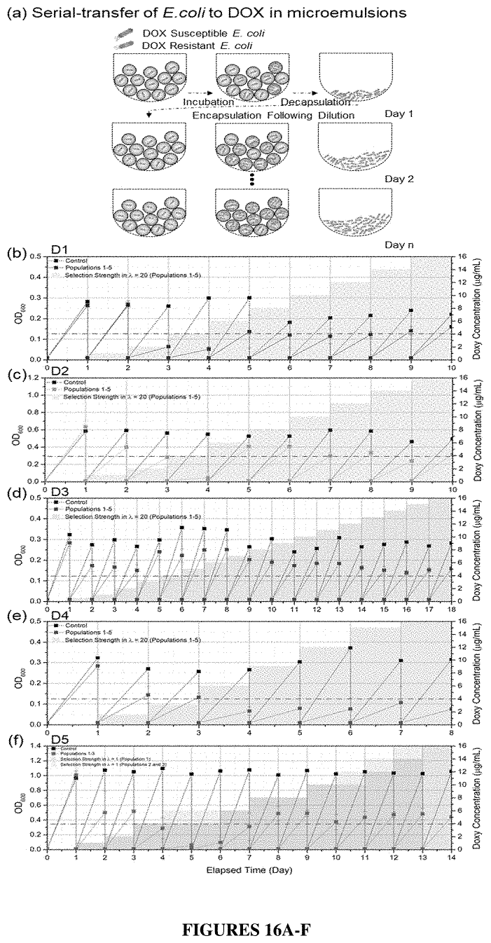

b) Serial-transfer of E. coli to DOX in microemulsions with a λ=20 following the same selection gradient as the serial-transfer protocol in flasks (D1 and D2 in Table 2). Five populations of E. coli were encapsulated in 247 μm microdroplets and adapted to DOX over 10 days using a λ=20 with two incubation conditions: 1.5 ml microfuge tube (D1) and 50 ml conical tube (D2). D2 offered improved aeration due to increased surface area at the air-liquid interface and higher headspace volume. A sixth no-drug control population without DOX was also passaged. The starting population size was ˜2.6×10⁶ cells/ml and could permit ˜6-8 doublings (FIG. 16B, D1 and D2) before the carrying capacity was reached. However, it was clear that at least some of the populations did not reach carrying capacity since cells growing in 1.5 ml tubes (D1) in the absence of DOX reached OD values of 0.3 after 24 hours of incubation, which was lower compared to cells growing in 50 ml tubes (D2; OD=0.6). As seen in FIGS. 16A-F on Days 2-4 the OD of DOX exposed populations was ˜6-fold lower than is typical for a suspension culture or the no-drug control population in microdroplets. All five populations within microemulsions in D1 and D2 showed a significant decrease in growth leading to population decrease as the DOX concentration approached the MIC (4 μg/ml). In the absence of antibiotic there was no significant decrease in population over time. A DOX gradient that was comfortably tolerated in flasks caused growth arrest in microdroplets, suggesting that expansion of a single genotype was limited by the carrying capacity of a microdroplet that led to the population decreases whereas a single genotype could grow out to full cell density in a suspension culture experiment.

c) Serial-transfer of E. coli to DOX in microemulsions with varying DOX selection gradients (D3 and D4). As before, five populations were established for each condition at a λ=20 and encapsulated in 247 μm microdroplets to provide a starting population of ˜2.6×10⁶ cells/ml. A weaker selection gradient, 0.5× the gradient established by flask transfer (FIGS. 16D, D3), took 18 days and showed no signs of a population decrease, suggesting that the weaker selection successfully attenuated the greater stress on the population that was observed in the 10 (normal gradient-D1), 8 (moderate gradient-D4) and 6 (strong gradient-D6) day adaptation schedules. Using a moderately stronger gradient, 1.5× stronger than that established by flask transfer (FIG. 16E, D4), the inventors were successful in passaging two of the five populations over 8 days instead of 10. Three of the five populations went to extinction while the remaining two were able to reach a final cell density above 0.05 OD/ml. The two populations that completed the 8 day DOX gradient reached a final cell density ˜4 times lower than the no-drug control population, which is consistent with the increased strength of the selection gradient. A DOX gradient that was 2× stronger than the gradient established by flask transfer (strong gradient, D6 in Table 2) and sought to adapt the populations to twice the DOX clinical breakpoint (16 μg/ml) over 6 days instead of 10 was unsuccessful as all five populations went to extinction.

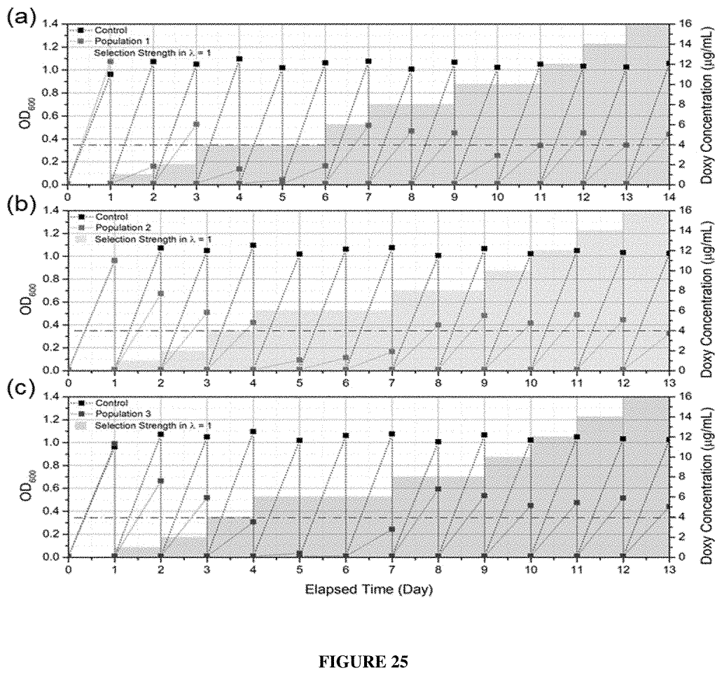

d) Serial-transfer of E. coli to DOX in microemulsions with a λ=1 (D5). Again, three populations of E. coli were encapsulated into 93 μm microdroplets with λ=1 in a 50 ml incubation vessel and adapted to DOX using an antibiotic selection gradient identical to that used for the serial transfer experiment (FIGS. 24A-B). A fourth control population without DOX was also serially transferred in λ=1 microemulsions. As before, each population had a starting size of ˜2.6×10⁶ cells/ml. The carrying capacity of 93 μm microdroplets allows for ˜6-8 doublings. Populations growing in the absence of DOX (black line in FIG. 16F) grew to a much higher final OD, suggesting improved growth in these droplets. It is important to note that unlike flask transfer, during each passage in the microdroplets, the parameter kept constant was λ value, not the dilution factor. Thus, the population was diluted to achieve the same starting λ value, which gave the population an opportunity to undergo a similar number of doublings every day. However, the λ=1 populations showed significantly greater population loss than either the serial transfer in flasks or encapsulated populations with λ=20 using the same DOX gradient (FIG. 16F and FIG. 25). The decrease in growth was much greater with λ=1 and occurred earlier and more frequently than when using a λ=20 (FIG. 16C).

Growth characteristics of E. coli within microemulsions. Microemulsions provide a substantially different growth environment from well-mixed suspension culture populations within flasks. Microemulsions cannot be agitated strongly without causing microdroplets to coalesce. Without strong agitation, aeration within microemulsions is dependent on diffusion across the oil and the surface area exposed to atmosphere. While the surface area can be increased, larger surface areas also produce dehydration of microdroplets.

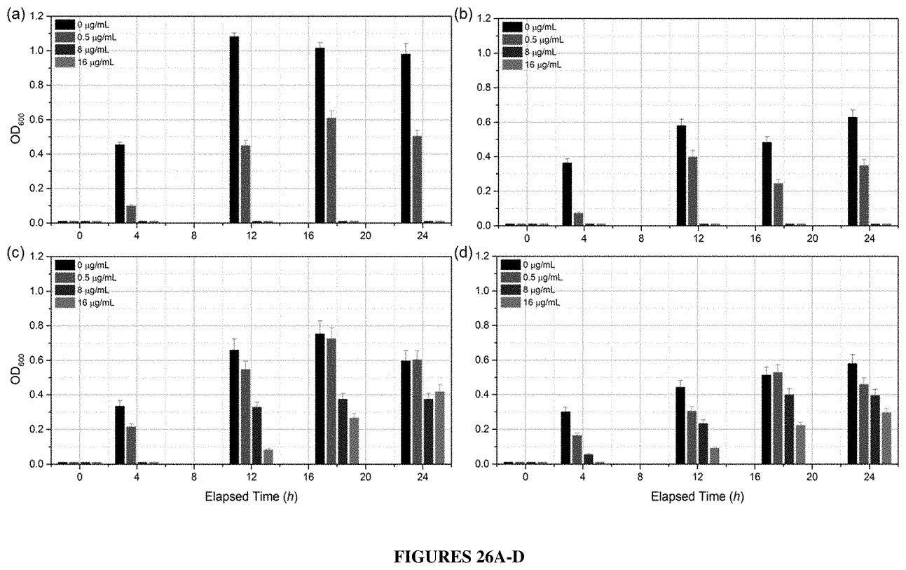

Initial experiments conducted by incubating cells encapsulated in microdroplets in 1.5 ml microfuge tubes suggested that aeration through the oil was potentially limiting as the final OD was typically ˜0.3 OD/ml (FIG. 16B)^{30,31} significantly less than is typical in a well aerated suspension culture. The inventors found that a good combination of droplet stability, growth rate and final population size were obtained when microemulsions were incubated in a 50 ml tube with a larger gas headspace (FIGS. 16B-C). Even though they provided a larger headspace for better aeration, in the absence of antibiotic, the inventors found that OD₆₀₀ of wild type E. coli at 24 h in 247 μm microdroplets was ˜0.6, a decrease of about ˜40% from that obtained under bulk growth conditions of a flask (FIGS. 26A-B). When the droplet size was reduced to 93 μm, 1 ml of emulsion could accommodate ˜2e6 number of droplets and after 24 h of growth, was able to reach an OD of 1.0 (FIG. 16F). Without sufficient aeration, the bacterial density was found to be lower in droplets resting at the bottom of the tube than those at the surface. As a result, depending on the specific conditions, such as the surface area of microemulsions exposed to air and volume of the emulsions, the number of generations of cells within droplets can be variable. Using a combination of a larger headspace and smaller 93 μm microdroplets, the inventors were able to achieve growth comparable to well mixed suspension cultures.

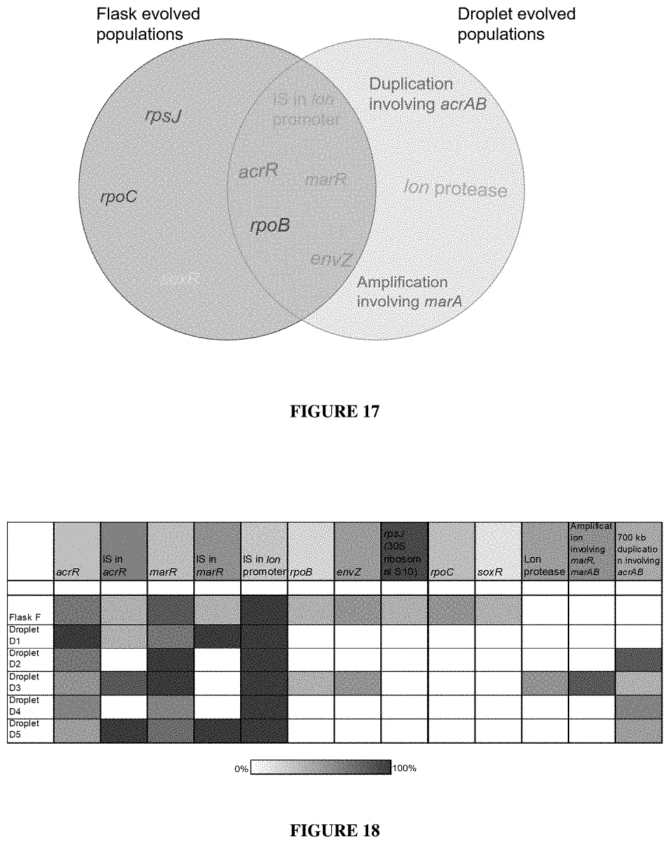

Evolutionary trajectories identified from the canonical serial transfer and microdroplet emulsion studies correctly identified the genetic drivers to DOX resistance, but also suggest that microdroplet environments may favor alternative genetic mechanisms to adaptation. At the end of the experiment, individual end-point isolates were isolated randomly by streaking the entire population onto non-selective, nutrient-rich agar plates. End point isolates are defined as randomly selected, independent colonies derived from an evolved population. Since evolved populations are diverse, each end point isolate could potentially have different genotypes. A minimum of three end-point isolates was subjected to whole genome sequencing from each experimental evolution study. In addition, total genomic DNA for each evolved population was isolated and subjected to metagenomic analysis to identify the allelic frequency in the final populations (Dataset S1-S6). The populations were sequenced to achieve at least 100× coverage and a threshold of 5% was used to identify mutations. Both serial transfer and microdroplet populations identified the well-known adaptive alleles marR, acrR, rpsJ and envZ³²⁻³⁵ as well as the movement of an insertion element IS186 into the upstream regulatory region of the gene encoding the Lon protease (FIG. 17). The AcrAB-TolC transporter has been shown previously to be upregulated in response to members of the tetracycline class of antibiotics in Gram negative pathogens by acquiring mutations in the repressors, AcrR and MarR^{34,36,37} Disruption or downregulation of Ion also leads to increased steady-state levels of AcrAB-TolC and low-levels of tetracycline resistance by decreasing degradation of the MarA activator³⁷. Both methods clearly implicated regulation of the AcrAB-TolC as the fundamental driver for DOX resistance, but there were interesting differences in the evolutionary trajectories identified.

Genomic re-arrangements in populations and isolates evolved using flask transfer utilized movement of insertion elements into the acrR and marR coding regions as well as in the upstream regulatory region of lon. In populations and isolates evolved using microdroplets, more sophisticated and substantial genomic re-arrangements were observed. In addition to movement of insertion elements, two other interesting events occurred: (1) duplication of a large region of the genome (˜700 kb) that was flanked by direct repeats and encoding acrAB and (2) a single novel junction that resulted from the 10-fold amplification of a 52 kb region of the genome. The amplified genetic element contained the marR gene and marAB operon. Both adaptive trajectories increased the copy number of genes involved in DOX resistance, suggesting that these events directly contributed to increased resistance and were only found in the microdroplet environment.

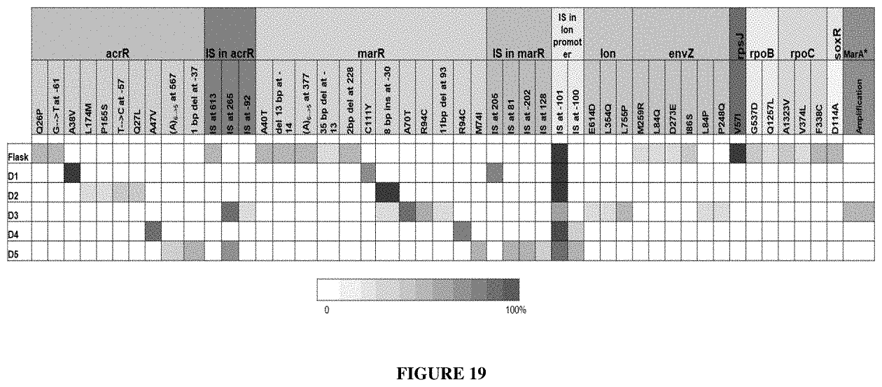

Amplification of genomic regions as an adaptive strategy has been observed clinically as well as in in vitro evolution experiments. Transposon or insertion sequence mediated amplification of beta-lactamase genes and tetM have contributed to carbapenem and tigecycline resistance, respectively^{15,25}. Tandem amplification of part of the citrate fermentation operon during experimental evolution gave E. coli the ability to aerobically utilize citrate³⁸. In the present study, the 700 kb duplication identified in the evolved population was a tandem amplification mediated by two direct repeats of IS3 flanking the amplified region. Similar duplication events involving different insertion elements have been documented in E. coli isolates recovered from agar plates supplemented with tetracycline³⁵. The 700 kb duplication was evident from a doubling of the sequencing depth. Given that it was mediated by direct IS3 repeats, no new junction evidence was identified in these populations. It is difficult to say if this amplification occurred in a sub-population or saturated the entire population, but it is clear that it was reproducibly seen in multiple replicate populations across different droplet conditions (FIGS. 18 and 19, D2-D5).

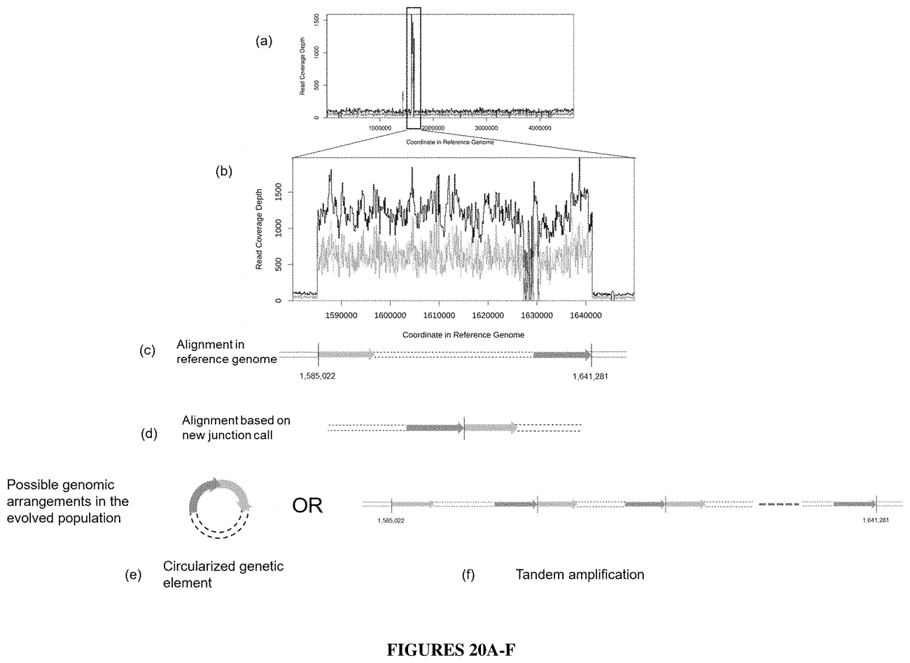

The 52 kb amplification became evident because of a large increase in coverage in that region and a new junction that was observed in the evolved population, but was absent in the ancestor. This pattern supports two possibilities: a circularization and amplification event or a tandem amplification event (FIGS. 20A-F). Interestingly, the ends of this 52 kb region have the XerCD dif sites which are involved in RecA independent recombination³⁹ and part of the 52 kb region encodes a cryptic prophage. Also encoded in this region are the genes marR, marA and marB. While the precise mechanism of the amplification remains to be determined, it is clear that in the context of DOX resistance, this amplification event likely serves the purpose of increasing the copy number of the gene encoding the efflux pump activator, marA.

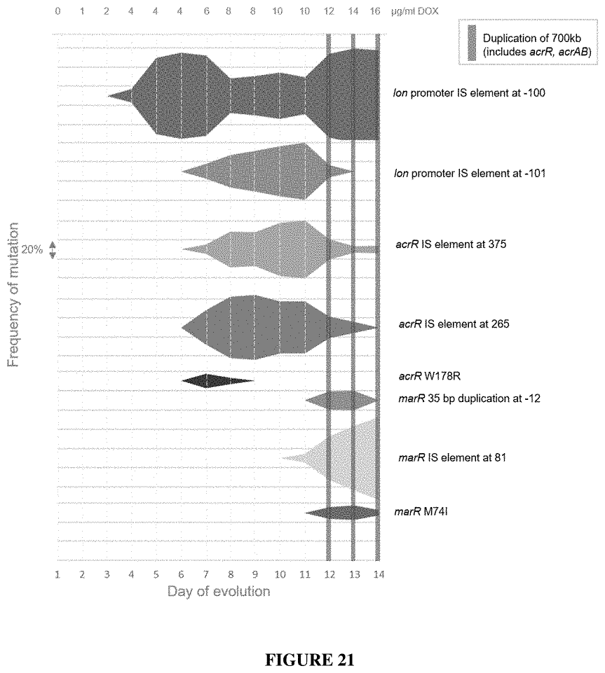

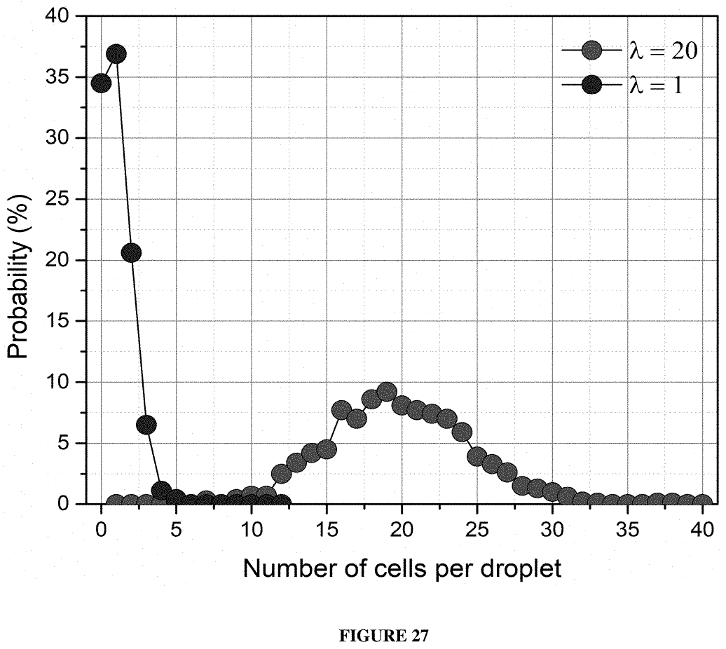

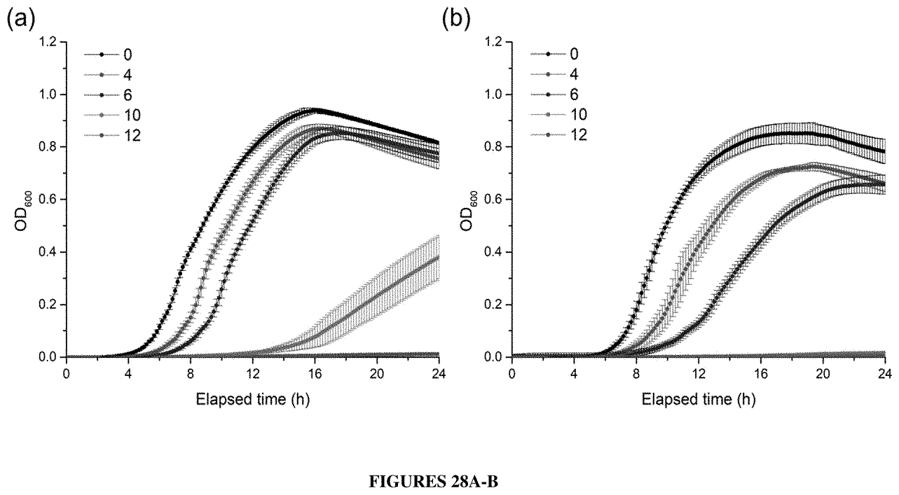

Longitudinal metagenomic study of a λ-1 microemulsion population. One challenge to conducting experimental evolution in microemulsions is the much smaller volumes used which could limit the founding population size. Using the microfluidic devices developed for this study, the inventors could readily produce microemulsions of sufficient volume (1 ml) and rapidity for daily surveys of a population under selection. They performed a longitudinal study to quantitate the rise and fall of adaptive alleles of a λ=1 population over time (Dataset S7). As shown in FIG. 21, after the initial success of early adaptive mutations in lon, acrR mutants achieved great success and it was not until DOX rose above 10 m/ml that mutations within marR were observed that then replaced acrR genotypes. Isolates with either acrR or marR confer resistance at lower DOX concentrations and have comparable growth rates (FIGS. 28A-B) and thus, the inventors anticipated that both acrR and marR mutants would be represented early in the experiment. They have no evidence that the mutation supply is strongly different in either evolutionary trajectory. For example, adaptive SNPs were seen for both acrR (AcrR^{W178R}) and marR (MarR^{M74I}) with the same rise and fall population dynamics as the more successful genomic rearrangements (FIG. 21). Since the λ=1 value is an average across microdroplets, there is still a subpopulation of microdroplets with λ>1 (29.3%, FIG. 27) allowing some opportunity for clonal interference if the early acrR containing genotypes have an as yet unknown fitness advantage in microemulsions such as better growth at lower levels of aeration or in smaller population sizes. The current study has not identified conditions within the microemulsions that favor acrR over marR and are the subject of further studies.

Is the mutational landscape underlying the accessible pathways to resistance through marR and acrR being undersampled? Aggregating the mutation data for the adaptive changes of acrR and marR across all the experiments (Datasets S1-S8), it is clear that the specific SNPs, indels and larger genetic changes that are the result of amplification or duplication can vary with each experimental evolution condition. These results suggest that despite having a starting population size of ˜2e6 each day, the 25 populations across all the methods showed little evidence of convergence to a single set of evolutionary trajectories with the exception of consistently identifying acrR and marR as critical targets for adaptation. This is not unexpected as the most advantageous adaptations to DOX resistance are those that reduce the function of AcrR and MarR. There are a very large number of mutations that could reduce function as compared to those that might improve function or activity. For example, the insertion of IS186 occurred within a very specific upstream region of Ion suggesting that the insertion element integration may have had a much higher frequency. It is also possible that other adaptive changes that could have happened, such as a frameshift early in the gene, might have had higher fitness costs. The former appears more plausible given that the Ion promoter has been identified as a hotspot for IS186 insertion⁴⁰. In addition, it has been observed that Ion mutations impart genome instability and higher levels of IS transpositions³⁵. Based on this, it was not surprising to see IS element mediated disruption of acrR and marR in both flask and microdroplet evolved populations. It was, however, unexpected to see other genomic re-arrangements (as described above) only in the droplet evolved populations but not in flasks. Like other experimental evolution approaches, the microdroplet environment may favor particular evolutionary trajectories that are not readily observed by other methods^{41,42}.

TABLE 2
Six experimental evolution studies for adaptation of E. coli BW25113 to DOX

| | Volume | | Droplets to | | | | OD | | Time taken |
|---|---|---|---|---|---|---|---|---|---|
| | of incuba- | Incuba- | headspace | Droplet | | | of the | Selection | to grow at |
| | bation | tion con- | volume | size | λ | Carrying | control | gradient | 16 μg/ml |
| Condition | vessel | dition | ratio | (μm) | value | capacity | population | strength | DOX |
| Flask (F) | 75 ml | Shaking | N/A | N/A | N/A | N/A | ~1.0 | Normal | 10 days |
| Droplet D1 | 1.5 ml | Static | 1:0.06 | 247 | 20 | ~1600 | ~0.3 | Normal | 10 days |
| Droplet D2 | 50 ml | Static | 1:33 | 247 | 20 | ~1600 | ~0.6 | Normal | 10 days |
| Droplet D3 | 1.5 ml | Static | 1:0.06 | 247 | 20 | ~1600 | ~0.3 | Weak | 18 days |
| Droplet D4 | 1.5 ml | Static | 1:0.06 | 247 | 20 | ~1600 | ~0.3 | Moderate | 8 days |
| Droplet D5 | 50 ml | Static | 1:33 | 93 | 1 | ~130 | ~1.0 | Normal | 13-14 days |
| Droplet D6 | 1.5 ml | Static | 1:0.06 | 247 | 20 | ~1600 | ~0.3 | Strong | 6 days* |

*Adaptation was scheduled to take 6 days but all populations went extinct.

TABLE 3
Minimum inhibitory concentration (MIC, μg/ml) of DOX for end-point isolates
obtained from six experimental evolution studies for adaptation of E. coli BW25113 to
DOX in batch (F) and microemulsions (D1-D5)

| | C1 | C2 | C3 | 1-1 | 1-2 | 1-3 | 2-1 | 2-2 | 2-3 | 3-1 | 3-2 | 3-3 | 4-1 | 4-2 | 4-3 | 5-1 | 5-2 | 5-3 |
|---|---|---|---|---|---|---|---|---|---|---|---|---|---|---|---|---|---|---|
| F | 4 | 4 | 4 | >16 | 16 | 16 | 16 | 16 | >16 | 16 | 16 | 16 | 16 | 16 | >16 | 16 | >16 | >16 |
| D1 | 4 | 4 | 4 | 8 | 8 | 8 | 16 | 16 | 16 | 16 | 16 | 16 | 16 | >16 | 16 | 16 | 16 | 16 |
| D2 | 4 | 4 | 4 | 16 | 16 | 16 | 16 | 16 | 16 | 16 | 16 | 16 | 16 | 16 | 16 | 16 | 16 | 16 |
| D3 | 4 | 4 | 4 | 16 | 16 | 16 | 8 | 16 | 8 | 16 | 8 | 16 | 16 | 16 | 16 | 16 | 16 | 16 |
| D4 | 4 | 4 | 4 | | | | | | | | | | 16 | 16 | 16 | 16 | 16 | 8 |
| D5 | 4 | 4 | 4 | 16 | >16 | 16 | >16 | 16 | 16 | >16 | 16 | >16 | | | | | | |

Complete genotypes of these isolates can be found in Dataset S8.
C1, C2 and C3 represent isolates obtained from populations evolved in the absence of DOX (no drug control) for each selection condition.
Nomenclature for isolate names: Number before dash represents the replicate population from which the isolate was obtained. The number after the dash represents the isolate number.

### Example 4—Discussion

In vitro experimental evolution has proven to be a remarkably effective method for the identification of biomarkers and the elucidation of adaptive trajectories responsible for new phenotypes. Serial transfer methods have been the principal approach to experimental evolution since they were first popularized by Lenski and co-workers in their seminal long-term experimental evolution work beginning in the 1990s⁴³. Since then, a wealth of studies have come out defining the strengths, weaknesses, methodological limits and interpretation of data from serial transfer studies^{5,9,44}. The inventors show that flow-focusing microfluidic chips can very rapidly produce homogeneous microdroplets suitable for experimental evolution allowing excellent control of the carrying capacity and number of cells per microdroplet (λ) and that suitable aeration of microdroplets can be achieved. Taken together, the control of microemulsion size and composition allows investigators to tune selection and population dynamics to address much more specific sets of questions compared to suspension culture methods. The evolutionary trajectories identified in this study highlight the prominent genetic drivers to DOX resistance but also show a stronger and consistent tendency across conditions and populations to favor quite unusual genomic rearrangements in getting there. It is still to be determined whether a tendency to select for large genomic re-arrangements is general to microemulsions due to changes in their microenvironment or is specific to the case of DOX adaptation in E. coli. With increases in the ability to perform long-read sequencing there has been increased recognition that substantive genomic rearrangements and transient amplification of regions may be under recognized in both the lab and clinic^{15,45}.

Evolutionary dynamics inside microdroplets are affected by various factors. Some of them include droplet size (i.e., carrying capacity), starting number of cells (λ value) and strength of selection. The number of doublings achieved depends on both droplet size and λ and so does the dilution factor. Thus, carefully selecting these parameters while designing the experiment will be necessary. There will most likely be some trade-offs involved. For example, in order to achieve a very low λ value and ensure that each droplet encapsulates a maximum of 1 cell, one has to compromise on starting population size or start with a very large emulsion volume which can be costly and time consuming. Unlike a suspension culture experiment, expansion of an adaptive genotype will be limited by the carrying capacity of the microdroplet, which likely explains why the rise of successful genotypes was consistently slower (as seen by the stalling of some populations in FIGS. 16A-F). Among the strengths of microfluidic approaches is the ability to sort individual microdroplets into plates using downstream microfluidic devices to determine genotypes or the diversity of genotypes (using single cell approaches) within microdroplets.

To explore the potential of microemulsions experimental evolution, the inventors developed and tested devices to produce highly monodisperse microdroplets for serial propagation. Earlier studies employed polydisperse microdroplets to produce sufficient volumes of emulsions but high volume variation produces very disparate λ values that limit tunability^{7,46}. They chose to change microdroplet diameters through fine modulation of microchannel geometries in order to explore different λ values. The precise controllability for the starting number of cells within each microdroplet could be achieved through the utilization of monodisperse microdroplets with low volume variation. Furthermore, a scale-up of microfluidic droplets generator can be achieved via parallelization while maintaining high uniformity of microdroplets.

With the arrival of microdroplet approaches to microbiology new areas of investigation have been opened up that allow experimenters exquisite control of molecular evolution studies in many interesting ways. Microdroplet-based experimental evolution provides opportunities for producing spatial structure that can be important to the study of social interactions in microbial communities, development of new applications in biotechnology, and biomarker discovery. As in the early days of serial transfer-based experimental evolution, the field is still early in understanding how microdroplets can be applied for the study of molecular evolution.

In sum, the inventors have successfully designed an experimental evolution platform that uses monodisperse microdroplets created using an optimized microdroplet generator that is capable of parallelization and have applied it to identify evolutionary trajectories associated with antimicrobial resistance. Furthermore, they evaluated how changes in key parameters such as droplet size, the starting number of cells within each microdroplet, selection gradients, and incubation method affect the evolutionary trajectories accessible to E. coli during adaptation to DOX. The results presented here show that this platform can allow exquisite control of these key parameters and can affect evolutionary dynamics within microdroplets. The inventors also observed that microdroplets in experimental evolution successfully identified not only adaptive alleles to the tetracycline class of antibiotics, but also a series of new adaptive pathways that alter the genomic structure. These findings can help in developing high-throughput experimental evolution approaches for novel biomarker discovery.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.

### For Background and Example 1

1. de Kraker, M. E.; Stewardson, A. J.; Harbarth, S., Will 10 Million People Die a Year due to Antimicrobial Resistance by 2050? PLoS Med 2016, 13 (11), e1002184.
2. Laupland, K. B.; Ruppe, E.; Harbarth, S., In 2035, will all bacteria be multidrug resistant? We are not sure. Intensive Care Med 2016, 42 (12), 2021-2023.
3. Antibiotic Resistance Threats in the United States, 2019. Centers for Disease Control and Prevention 2019.
4. WHO publishes list of bacteria for which new antibiotics are urgently needed. 2017.
5. O'Neill, J., Tackling Druf-Resisitant Infections: Final Report and Recommendations. The Review on Antimicrobial Resistance 2016.
6. Boucher, H. W.; Talbot, G. H.; Bradley, J. S.; Edwards, J. E.; Gilbert, D.; Rice, L. B.; Scheld, M.; Spellberg, B.; Bartlett, J., Bad bugs, no drugs: no ESKAPE! An update from the Infectious Diseases Society of America. Clinical infectious diseases: an official publication of the Infectious Diseases Society of America 2009, 48 (1), 1-12.
7. Rice, L. B., Federal funding for the study of antimicrobial resistance in nosocomial pathogens: no ESKAPE. J Infect Dis 2008, 197 (8), 1079-81.
8. Berdy, J., Thoughts and facts about antibiotics: Where we are now and where we are heading. J Antibiot (Tokyo) 2012, 65 (8), 441.
9. Watve, M. G.; Tickoo, R.; Jog, M. M.; Bhole, B. D., How many antibiotics are produced by the genus Streptomyces? Arch Microbiol 2001, 176 (5), 386-90.
10. D. A., H., Streptomyces in nature and medicine: the antibiotic makers. Oxford University Press: 2007.
11. Liu, G.; Chater, K. F.; Chandra, G.; Niu, G.; Tan, H., Molecular regulation of antibiotic biosynthesis in streptomyces. Microbiology and molecular biology reviews: MMBR 2013, 77 (1), 112-43.
12. Bibb, M. J., Regulation of secondary metabolism in streptomycetes. Curr Opin Microbiol 2005, 8 (2), 208-15.
13. Bachmann, H.; Fischlechner, M.; Rabbers, I.; Barfa, N.; Branco dos Santos, F.; Molenaar, D.; Teusink, B., Availability of public goods shapes the evolution of competing metabolic strategies. Proc Natl Acad Sci USA 2013, 110 (35), 14302-7.
14. van Tatenhove-Pel, R. J.; Hernandez-Valdes, J. A.; Teusink, B.; Kuipers, 0. P.; Fischlechner, M.; Bachmann, H., Microdroplet screening and selection for improved microbial production of extracellular compounds. Curr Opin Biotechnol 2020, 61, 72-81.
15. Jones, S. E.; Elliot, M. A., Streptomyces Exploration: Competition, Volatile Communication and New Bacterial Behaviours. Trends in microbiology 2017, 25 (7), 522-531.
16. Jones, S. E.; Ho, L.; Rees, C. A.; Hill, J. E.; Nodwell, J. R.; Elliot, M. A., Streptomyces exploration is triggered by fungal interactions and volatile signals. Elife 2017, 6.
17. Prater, A. G.; Mehta, H. H.; Beabout, K.; Supandy, A.; Miller, W. R.; Tran, T. T.; Arias, C. A.; Shamoo, Y., Daptomycin Resistance in Enterococcus faecium Can Be Delayed by Disruption of the LiaFSR Stress Response Pathway. Antimicrob Agents Chemother 2021, 65 (4).
18. Rincon, S.; Panesso, D.; Miller, W. R.; Singh, K. V.; Cruz, M. R.; Khan, A.; Dinh, A. Q.; Diaz, L.; Rios, R.; Shamoo, Y.; Reyes, J.; Tran, T. T.; Garsin, D. A.; Arias, C. A., Disrupting Membrane Adaptation Restores In Vivo Efficacy of Antibiotics Against Multidrug-Resistant Enterococci and Potentiates Killing by Human Neutrophils. J Infect Dis 2019, 220 (3), 494-504.
19. Prater, A. G.; Mehta, H. H.; Kosgei, A. J.; Miller, W. R.; Tran, T. T.; Arias, C. A.; Shamoo, Y., Environment Shapes the Accessible Daptomycin Resistance Mechanisms in Enterococcus faecium. Antimicrob Agents Chemother 2019, 63 (10).
20. Miller, W. R.; Tran, T. T.; Diaz, L.; Rios, R.; Khan, A.; Reyes, J.; Prater, A. G.; Panesso, D.; Shamoo, Y.; Arias, C. A., LiaR-independent pathways to daptomycin resistance in Enterococcus faecalis reveal a multilayer defense against cell envelope antibiotics. Molecular microbiology 2019, 111 (3), 811-824.
21. Davlieva, M.; Wu, C.; Zhou, Y.; Arias, C. A.; Shamoo, Y., Two Mutations Commonly Associated with Daptomycin Resistance in Enterococcus faecium LiaS(T120A) and LiaR(W73C) Appear To Function Epistatically in LiaFSR Signaling. Biochemistry 2018.
22. Davlieva, M.; Tovar-Yanez, A.; DeBruler, K.; Leonard, P. G.; Zianni, M. R.; Arias, C. A.; Shamoo, Y., An Adaptive Mutation in Enterococcus faecium LiaR Associated with Antimicrobial Peptide Resistance Mimics Phosphorylation and Stabilizes LiaR in an Activated State. J Mol Biol 2016, 428 (22), 4503-4519.
23. Reyes, J.; Panesso, D.; Tran, T. T.; Mishra, N. N.; Cruz, M. R.; Munita, J. M.; Singh, K. V.; Yeaman, M. R.; Murray, B. E.; Shamoo, Y.; Garsin, D.; Bayer, A. S.; Arias, C. A., A liaR deletion restores susceptibility to daptomycin and antimicrobial peptides in multidrug-resistant Enterococcus faecalis. J Infect Dis 2015, 211 (8), 1317-25.
24. Panesso, D.; Reyes, J.; Gaston, E. P.; Deal, M.; Londono, A.; Nigo, M.; Munita, J. M.; Miller, W. R.; Shamoo, Y.; Tran, T. T.; Arias, C. A., Deletion of liaR Reverses Daptomycin Resistance in Enterococcus faecium Independent of the Genetic Background. Antimicrob Agents Chemother 2015, 59 (12), 7327-34.
25. Diaz, L.; Tran, T. T.; Munita, J. M.; Miller, W. R.; Rincon, S.; Carvajal, L. P.; Wollam, A.; Reyes, J.; Panesso, D.; Rojas, N. L.; Shamoo, Y.; Murray, B. E.; Weinstock, G. M.; Arias, C. A., Whole-genome analyses of Enterococcus faecium isolates with diverse daptomycin MICs. Antimicrob Agents Chemother 2014, 58 (8), 4527-34.
26. Tran, T. T.; Panesso, D.; Mishra, N. N.; Mileykovskaya, E.; Guan, Z.; Munita, J. M.; Reyes, J.; Diaz, L.; Weinstock, G. M.; Murray, B. E.; Shamoo, Y.; Dowhan, W.; Bayer, A. S.; Arias, C. A., Daptomycin-resistant Enterococcus faecalis diverts the antibiotic molecule from the division septum and remodels cell membrane phospholipids. MBio 2013, 4 (4).
27. Tran, T. T.; Panesso, D.; Gao, H.; Roh, J. H.; Munita, J. M.; Reyes, J.; Diaz, L.; Lobos, E. A.; Shamoo, Y.; Mishra, N. N.; Bayer, A. S.; Murray, B. E.; Weinstock, G. M.; Arias, C. A., Whole-genome analysis of a daptomycin-susceptible Enterococcus faecium strain and its daptomycin-resistant variant arising during therapy. Antimicrob Agents Chemother 2013, 57 (1), 261-8.
28. Miller, C.; Kong, J.; Tran, T. T.; Arias, C. A.; Saxer, G.; Shamoo, Y., Adaptation of Enterococcus faecalis to daptomycin reveals an ordered progression to resistance. Antimicrob Agents Chemother 2013, 57 (11), 5373-83.
29. Davlieva, M.; Zhang, W.; Arias, C. A.; Shamoo, Y., Biochemical characterization of cardiolipin synthase mutations associated with daptomycin resistance in enterococci. Antimicrob Agents Chemother 2013, 57 (1), 289-96.
30. Mishra, N. N.; Bayer, A. S.; Tran, T. T.; Shamoo, Y.; Mileykovskaya, E.; Dowhan, W.; Guan, Z.; Arias, C. A., Daptomycin resistance in enterococci is associated with distinct alterations of cell membrane phospholipid content. PLoS One 2012, 7 (8), e43958.
31. Arias, C. A.; Panesso, D.; McGrath, D. M.; Qin, X.; Mojica, M. F.; Miller, C.; Diaz, L.; Tran, T. T.; Rincon, S.; Barbu, E. M.; Reyes, J.; Roh, J. H.; Lobos, E.; Sodergren, E.; Pasqualini, R.; Arap, W.; Quinn, J. P.; Shamoo, Y.; Murray, B. E.; Weinstock, G. M., Genetic basis for in vivo daptomycin resistance in enterococci. N Engl J Med 2011, 365 (10), 892-900.
32. Hammerstrom, T. G.; Beabout, K.; Clements, T. P.; Saxer, G.; Shamoo, Y., Acinetobacter baumannii Repeatedly Evolves a Hypermutator Phenotype in Response to Tigecycline That Effectively Surveys Evolutionary Trajectories to Resistance. PLoS One 2015, 10 (10), e0140489.
33. Mehta, H. H.; Prater, A. G.; Beabout, K.; Elworth, R. A. L.; Karavis, M.; Gibbons, H. S.; Shamoo, Y., The Essential Role of Hypermutation in Rapid Adaptation to Antibiotic Stress. Antimicrob Agents Chemother 2019, 63 (7).
34. Choi, K. H.; Schweizer, H. P., mini-Tn7 insertion in bacteria with secondary, non-glmS-linked attTn7 sites: example Proteus mirabilis H14320. Nature protocols 2006, 1 (1), 170-8.
35. van Aartsen, J. J.; Rajakumar, K., An optimized method for suicide vector-based allelic exchange in Klebsiella pneumoniae. Journal of microbiological methods 2011, 86 (3), 313-9.
36. Lazarus, J. E.; Warr, A. R.; Kuehl, C. J.; Giorgio, R. T.; Davis, B. M.; Waldor, M. K., A New Suite of Allelic-Exchange Vectors for the Scarless Modification of Proteobacterial Genomes. Appl Environ Microbiol 2019, 85 (16).

### For Examples 2-4

(1) de Kraker, M. E. A.; Stewardson, A. J.; Harbarth, S. Will 10 Million People Die a Year Due to Antimicrobial Resistance by 2050? PLOS Med. 2016, 13 (11), e1002184. /doi.org/10.1371/journal.pmed.1002184.
(2) Laupland, K. B.; Ruppé, E.; Harbarth, S. In 2035, Will All Bacteria Be Multidrug Resistant? We Are Not Sure. Intensive Care Med. 2016, 42 (12), 2021-2023. /doi.org/10.1007/s00134-016-4343-2.
(3) Centers for Disease Control and Prevention (U.S.). Antibiotic Resistance Threats in the United States, 2019; Centers for Disease Control and Prevention (U.S.), 2019. /doi.org/10.15620/cdc:82532.
(4) Boolchandani, M.; D'Souza, A. W.; Dantas, G. Sequencing-Based Methods and Resources to Study Antimicrobial Resistance. Nat. Rev. Genet. 2019. /doi.org/10.1038/s41576-019-0108-4.
(5) Barrick, J. E.; Lenski, R. E. Genome Dynamics during Experimental Evolution. Nat. Rev. Genet. 2013, 14 (12), 827-839. /doi.org/10.1038/nrg3564.
(6) Jasmin, J.-N.; Dillon, M. M.; Zeyl, C. The Yield of Experimental Yeast Populations Declines during Selection. Proc. R. Soc. B Biol. Sci. 2012, 279 (1746), 4382-4388. /doi.org/10.1098/rspb.2012.1659.
(7) Bachmann, H.; Fischlechner, M.; Rabbers, I.; Barfa, N.; Branco dos Santos, F.; Molenaar, D.; Teusink, B. Availability of Public Goods Shapes the Evolution of Competing Metabolic Strategies. Proc. Natl. Acad. Sci. 2013, 110 (35), 14302-14307. /doi.org/10.1073/pnas.1308523110.
(8) Bachmann, H.; Pronk, J. T.; Kleerebezem, M.; Teusink, B. Evolutionary Engineering to Enhance Starter Culture Performance in Food Fermentations. Curr. Opin. Biotechnol. 2015, 32, 1-7. /doi.org/10.1016/j.copbio.2014.09.003.
(9) Lukac̆us̆inová, M.; Bollenbach, T. Toward a Quantitative Understanding of Antibiotic Resistance Evolution. Curr. Opin. Biotechnol. 2017, 46, 90-97. /doi.org/10.1016/j.copbio.2017.02.013.
(10) McDonald, M. J. Microbial Experimental Evolution—a Proving Ground for Evolutionary Theory and a Tool for Discovery. EMBO Rep. 2019, 20 (8). /doi.org/10.15252/embr.201846992.
(11) van Tatenhove-Pel, R. J.; Hernandez-Valdes, J. A.; Teusink, B.; Kuipers, 0. P.; Fischlechner, M.; Bachmann, H. Microdroplet Screening and Selection for Improved Microbial Production of Extracellular Compounds. Curr. Opin. Biotechnol. 2020, 61, 72-81. /doi.org/10.1016/j.copbio.2019.10.007.
(12) Hsu, R. H.; Clark, R. L.; Tan, J. W.; Ahn, J. C.; Gupta, S.; Romero, P. A.; Venturelli, O. S. Microbial Interaction Network Inference in Microfluidic Droplets. Cell Syst. 2019, 9 (3), 229-242.e4. /doi.org/10.1016/j.cels.2019.06.008.
(13) Shapiro, H. M. Practical Flow Cytometry; Wiley-Liss: New York, 2007.
(14) Mahler, L.; Wink, K.; Beulig, R. J.; Scherlach, K.; Tovar, M.; Zang, E.; Martin, K.; Hertweck, C.; Belder, D.; Roth, M. Detection of Antibiotics Synthetized in Microfluidic Picolitre-Droplets by Various Actinobacteria. Sci. Rep. 2018, 8 (1), 13087. /doi.org/10.1038/s41598-018-31263-2.
(15) Shropshire, W. C.; Aitken, S. L.; Pifer, R.; Kim, J.; Bhatti, M. M.; Li, X.; Kalia, A.; Galloway-Peña, J.; Sahasrabhojane, P.; Arias, C. A.; Greenberg, D. E.; Hanson, B. M.; Shelburne, S. A. IS26-Mediated Amplification of BlaOXA-1 and BlaCTX-M-15 with Concurrent Outer Membrane Porin Disruption Associated with de Novo Carbapenem Resistance in a Recurrent Bacteraemia Cohort. J. Antimicrob. Chemother. 2021, 76 (2), 385-395. /doi.org/10.1093/jac/dkaa447.
(16) Seo, S.; Perez, G. A.; Tewari, K.; Comas, X.; Kim, M. Catalytic Activity of Nickel Nanoparticles Stabilized by Adsorbing Polymers for Enhanced Carbon Sequestration. Sci. Rep. 2018, 8 (1), 11786. /doi.org/10.1038/s41598-018-29605-1.
(17) Seo, S.; Mastiani, M.; Mosavati, B.; Peters, D. M.; Mandin, P.; Kim, M. Performance Evaluation of Environmentally Benign Nonionic Biosurfactant for Enhanced Oil Recovery. Fuel 2018, 234, 48-55. /doi.org/10.1016/j.fue1.2018.06.111.
(18) Seo, S.; Lages, B.; Kim, M. Catalytic CO2 Absorption in an Amine Solvent Using Nickel Nanoparticles for Post-Combustion Carbon Capture. J. CO2 Util. 2020, 36, 244-252. /doi.org/10.1016/j.jcou.2019.11.011.
(19) Lai, D.; Frampton, J. P.; Sriram, H.; Takayama, S. Rounded Multi-Level Microchannels with Orifices Made in One Exposure Enable Aqueous Two-Phase System Droplet Microfluidics. Lab. Chip 2011, 11 (20), 3551. /doi.org/10.1039/c1lc20560a.
(20) Mastiani, M.; Seo, S.; Jimenez, S. M.; Petrozzi, N.; Kim, M. M. Flow Regime Mapping of Aqueous Two-Phase System Droplets in Flow-Focusing Geometries. Colloids Surf Physicochem. Eng. Asp. 2017, 531, 111-120. /doi.org/10.1016/j.colsurfa.2017.07.083.
(21) Sartipzadeh, O.; Naghib, S. M.; Seyfoori, A.; Rahmanian, M.; Fateminia, F. S. Controllable Size and Form of Droplets in Microfluidic-Assisted Devices: Effects of Channel Geometry and Fluid Velocity on Droplet Size. Mater. Sci. Eng. C 2020, 109, 110606. /doi.org/10.1016/j.msec.2019.110606.
(22) Abbaspourrad, A.; Carroll, N. J.; Kim, S.-H.; Weitz, D. A. Polymer Microcapsules with Programmable Active Release/pubs.acs.org/doi/pdfplus/10.1021/ja401960f (accessed 2020-12-19). /doi.org/10.1021/ja401960f.
(23) Zhu, P.; Kong, T.; Lei, L.; Tian, X.; Kang, Z.; Wang, L. Droplet Breakup in Expansion-Contraction Microchannels. Sci. Rep. 2016, 6 (1), 21527. /doi.org/10.1038/srep21527.
(24) Chopra, I.; Roberts, M. Tetracycline Antibiotics: Mode of Action, Applications, Molecular Biology, and Epidemiology of Bacterial Resistance. Microbiol. Mol. Biol. Rev. MMBR 2001, 65 (2), 232-260; second page, table of contents. /doi.org/10.1128/MMBR.65.2.232-260.2001.
(25) Beabout, K.; Hammerstrom, T. G.; Wang, T. T.; Bhatty, M.; Christie, P. J.; Saxer, G.;

Shamoo, Y. Rampant Parasexuality Evolves in a Hospital Pathogen during Antibiotic Selection. Mol. Biol. Evol. 2015, 32 (10), 2585-2597. /doi.org/10.1093/molbev/msv133.
(26) Anes, J.; McCusker, M. P.; Fanning, S.; Martins, M. The Ins and Outs of RND Efflux Pumps in Escherichia Coli. Front. Microbiol. 2015, 6, 587. /doi.org/10.3389/fmicb.2015.00587.
(27) Beabout, K.; McCurry, M. D.; Mehta, H.; Shah, A. A.; Pulukuri, K. K.; Rigol, S.; Wang, Y; Nicolaou, K. C.; Shamoo, Y. Experimental Evolution of Diverse Strains as a Method for the Determination of Biochemical Mechanisms of Action for Novel Pyrrolizidinone Antibiotics. ACS Infect. Dis. 2017, 3 (11), 854-865. /doi.org/10.1021/acsinfecdis.7b00135.
(28) Mehta, H. H.; Prater, A. G.; Shamoo, Y. Using Experimental Evolution to Identify Druggable Targets That Could Inhibit the Evolution of Antimicrobial Resistance. J. Antibiot. (Tokyo) 2018, 71 (2), 279-286. /doi.org/10.1038/ja.2017.108.
(29) Mehta, H. H.; Prater, A. G.; Beabout, K.; Elworth, R. A. L.; Karavis, M.; Gibbons, H. S.; Shamoo, Y. The Essential Role of Hypermutation in Rapid Adaptation to Antibiotic Stress. Antimicrob. Agents Chemother. 2019, 63 (7), e00744-19. /doi.org/10.1128/AAC.00744-19.
(30) Sjostrom, S. L.; Bai, Y.; Huang, M.; Liu, Z.; Nielsen, J.; Joensson, H. N.; Andersson Svahn, H. High-Throughput Screening for Industrial Enzyme Production Hosts by Droplet Microfluidics. Lab. Chip 2014, 14 (4), 806-813. /doi.org/10.1039/c31c51202a.
(31) Chowdhury, M. S.; Zheng, W.; Kumari, S.; Heyman, J.; Zhang, X.; Dey, P.; Weitz, D. A.; Haag, R. Dendronized Fluorosurfactant for Highly Stable Water-in-Fluorinated Oil Emulsions with Minimal Inter-Droplet Transfer of Small Molecules. Nat. Commun. 2019, 10 (1), 4546. /doi.org/10.1038/s41467-019-12462-5.
(32) Hoeksema, M.; Jonker, M. J.; Brul, S.; ter Kuile, B. H. Effects of a Previously Selected Antibiotic Resistance on Mutations Acquired during Development of a Second Resistance in Escherichia Coli. BMC Genomics 2019, 20 (1), 284. /doi.org/10.1186/s12864-019-5648-7.
(33) Toprak, E.; Veres, A.; Michel, J.-B.; Chait, R.; Hartl, D. L.; Kishony, R. Evolutionary Paths to Antibiotic Resistance under Dynamically Sustained Drug Selection. Nat. Genet. 2012, 44 (1), 101-105. /doi.org/10.1038/ng.1034.
(34) Grossman, T. H. Tetracycline Antibiotics and Resistance. Cold Spring Harb. Perspect. Med. 2016, 6 (4), a025387. /doi.org/10.1101/cshperspect.a025387.
(35) Nicoloff, H.; Perreten, V.; Levy, S. B. Increased Genome Instability in Escherichia Coli Lon Mutants: Relation to Emergence of Multiple-Antibiotic-Resistant (Mar) Mutants Caused by Insertion Sequence Elements and Large Tandem Genomic Amplifications. Antimicrob. Agents Chemother. 2007, 51 (4), 1293-1303. /doi.org/10.1128/AAC.01128-06.
(36) Weston, N.; Sharma, P.; Ricci, V.; Piddock, L. J. V. Regulation of the AcrAB-TolC Efflux Pump in Enterobacteriaceae. Res. Microbiol. 2018, 169 (7), 425-431. /doi.org/10.1016/j.resmic 0.2017.10.005.
(37) Nicoloff, H.; Perreten, V; McMurry, L. M.; Levy, S. B. Role for Tandem Duplication and Lon Protease in AcrAB-TolC- Dependent Multiple Antibiotic Resistance (Mar) in an Escherichia Coli Mutant without Mutations in MarRAB or AcrRAB. J. Bacteriol. 2006, 188 (12), 4413-4423. /doi.org/10.1128/JB.01502-05.
(38) Blount, Z. D.; Barrick, J. E.; Davidson, C. J.; Lenski, R. E. Genomic Analysis of a Key Innovation in an Experimental Escherichia Coli Population. Nature 2012, 489 (7417), 513-518. /doi.org/10.1038/nature11514.
(39) Tecklenburg, M.; Naumer, A.; Nagappan, 0.; Kuempel, P. The Dif Resolvase Locus of the Escherichia Coli Chromosome Can Be Replaced by a 33-Bp Sequence, but Function Depends on Location. Proc. Natl. Acad. Sci. 1995, 92 (5), 1352-1356. /doi.org/10.1073/pnas.92.5.1352.
(40) saiSree, L.; Reddy, M.; Gowrishankar, J. IS186 Insertion at a Hot Spot in the Lon Promoter as a Basis for Lon Protease Deficiency of Escherichia Coli B: Identification of a Consensus Target Sequence for IS186 Transposition. J. Bacteriol. 2001, 183 (23), 6943-6946. /doi.org/10.1128/JB.183.23.6943-6946.2001.
(41) Prater, A. G.; Mehta, H. H.; Kosgei, A. J.; Miller, W. R.; Tran, T. T.; Arias, C. A.; Shamoo, Y. Environment Shapes the Accessible Daptomycin Resistance Mechanisms in Enterococcus Faecium. Antimicrob. Agents Chemother. 2019, 63 (10), e00790-19. /doi.org/10.1128/AAC.00790-19.
(42) Santos-Lopez, A.; Marshall, C. W.; Scribner, M. R.; Snyder, D. J.; Cooper, V. S. Evolutionary Pathways to Antibiotic Resistance Are Dependent upon Environmental Structure and Bacterial Lifestyle. eLife 2019, 8, e47612. /doi.org/10.7554/eLife.47612.
(43) Lenski, R. E. Experimental Evolution and the Dynamics of Adaptation and Genome Evolution in Microbial Populations. ISME J. 2017, 11 (10), 2181-2194. /doi.org/10.1038/ismej.2017.69.
(44) Wahl, L. M.; Gerrish, P. J. The Probability That Beneficial Mutations Are Lost in Populations with Periodic Bottlenecks. Evol. Int. J. Org. Evol. 2001, 55 (12), 2606-2610. /doi.org/10.1111/j.0014-3820.2001.tb00772.x.
(45) Schechter, L. M.; Creely, D. P.; Garner, C. D.; Shortridge, D.; Nguyen, H.; Chen, L.; Hanson, B. M.; Sodergren, E.; Weinstock, G. M.; Dunne, W. M.; van Belkum, A.; Leopold, S. R. Extensive Gene Amplification as a Mechanism for Piperacillin-Tazobactam Resistance in Escherichia Coli. mBio 2018, 9 (2), e00583-18. /doi.org/10.1128/mBio.00583-18.
(46) van Tatenhove-Pel, R. J.; Zwering, E.; Boreel, D. F.; Falk, M.; van Heerden, J. H.; Kes, M. B. M. J.; Kranenburg, C. I.; Botman, D.; Teusink, B.; Bachmann, H. Serial Propagation in Water-in-Oil Emulsions Selects for Saccharomyces Cerevisiae Strains with a Reduced Cell Size or an Increased Biomass Yield on Glucose. Metab. Eng. 2021, 64, 1-14. /doi.org/10.1016/j.ymben.2020.12.005.
(47) Deatherage, D. E.; Barrick, J. E. Identification of Mutations in Laboratory Evolved Microbes from Next-Generation Sequencing Data Using Breseq. Methods Mol. Biol. 2014, 1151, 165-188. /doi.org/10.1007/978-1-4939-0554-6_12.

1. A microenvironment comprising a single predator strain of an antibiotic-producing microbe and a single strain of a multi-drug resistant (MDR) bacterial pathogen.

2. The microenvironment of claim 1, wherein said microenvironment is a water-in-oil (W/O) microdroplet.

3. The microenvironment of claim 2, wherein said microdroplet (a) is about 10-200 μm in diameter or about 100 μm in diameter; and/or (b) comprises 10 or fewer than microbial cells, such as 1 or 2 predator strain cells and 1 to 10 MDR pathogen cells.

4. The microenvironment of claim 1, wherein said predator strain produces a fluorescent signal and/or said MDR bacterial pathogen produces a fluorescent signal.

5. The microenvironment of claim 4, wherein said predator strain produces a first fluorescent signal and said MDR bacterial pathogen produces a second fluorescent signal, said first and second fluorescent signals being optically distinguishable.

6. The microenvironment of claim 1, wherein said predator strain is a culturable bacterium is selected from the group of wild or cultured isolates belonging to the genus Streptomyces, Bacillus, or Nocardia or a fungus belonging to the genus Penicillium.

7. The microenvironment of claim 1, wherein said MDR bacterial pathogen is selected from the group consisting of Pseudomonas, Nocardia, Escherichia, Klebsiella, Staphylococcus, Acinetobacter, Francisella, or Enterococcus.

8. The microenvironment of claim 1, wherein said microenvironment is disposed in a bacterial growth chamber containing bacterial growth media.

9. The microenvironment of claim 8, wherein said bacterial growth chamber comprises additional microenvironments each comprising said predator strain and said MDR bacterial pathogen.

10. The microenvironment of claim 1, wherein said predator strain has been subjected to a mutagen, such as N-methyl-N′-nitro-N-nitrosoguanidine.

11. A method of co-culturing a single predator strain of an antibiotic-producing microbe and a single strain of a multi-drug resistant (MDR) bacterial pathogen comprising:
(a) microencapsulating a single predator strain of an antibiotic-producing microbe and a single strain of a MDR bacterial pathogen to create a microenvironment; and
(b) culturing said microenvironment in a bacterial growth chamber containing bacterial growth medium.

12. The method of claim 11, wherein said microenvironment is a water-in-oil (W/O) microdroplet, such as generated by mixing aqueous and oil phases, in particular being generated through microfluidic processing.

13. The method of claim 12, wherein said microdroplet (a) is about 10-200 μm in diameter or about 100 μm in diameter; and/or (b) comprises 10 or fewer than microbial cells, such as 1 or 2 predator strain cells and 1 or 2 MDR pathogen cells.

14. The method of claim 11, wherein said predator strain produces a fluorescent signal and/or said MDR bacterial pathogen produces a fluorescent signal.

15. The method of claim 14, wherein said predator strain produces a first fluorescent signal and said MDR bacterial pathogen produces a second fluorescent signal, said first and second fluorescent signals being optically distinguishable.

16. The method of claim 11, further comprising assessing the relative amounts of said predator strain and said MDR bacterial pathogen after culture.

17. The method of claim 16, further comprising isolating said microenvironment when the relative amount of predator strain present is greater than said MDR bacterial pathogen, such as by fluorescence activated sorting.

18. The method of claim 17, further comprising obtaining said predator strain from said isolated microenvironment.

19. The method of claim 11, wherein said predator strain is a culturable bacterium is selected from the group wild or cultured isolates belonging to the genus Streptomyces, Bacillus, or Nocardia or a fungus belonging to the genus Penicillium.

20. The method of claim 11, wherein said MDR bacterial pathogen is selected from the group consisting of Pseudomonas, Nocardia, Escherichia, Klebsiella, Staphylococcus, Acinetobacter, Francisella, or Enterococcus.

21. The method of claim 11, wherein said bacterial growth chamber comprises additional microenvironments each comprising said predator strain and said MDR bacterial pathogen.

22. The method of claim 11, wherein said predator strain has been subjected to a mutagen.

23. The method of claim 11, wherein culturing is performed for about 24 hours to about 72 hours, or for about 48 hours, optionally at 30° C.

24. The method of claim 17, further comprising:
(c) decapsulating said selected predator strain;
(d) re-microencapsulating said selected predator strain and a single strain of a MDR bacterial pathogen to create a second microenvironment;
(e) culturing said second microenvironment in a bacterial growth chamber containing bacterial growth medium;
(f) assessing the relative amounts of said predator strain and the MDR bacterial pathogen of step (d) after culture;
(g) isolating said second microenvironment when the relative amount of predator strain present is greater than said MDR bacterial pathogen; and
(h) obtaining said predator strain from said isolated second microenvironment.

25. The method of claim 24, wherein steps (c)-(h) are further repeated, such as for a total of 10 cycles.

26. The method of claim 25, wherein a further mutagenesis step is applied to the predator strain after one or more cycles.

27. The method of claim 25, further comprising changing microenvironment volume and/or ratio of said predator strain to said MDR bacterial pathogen between cycles.

28. The method of claim 11, wherein step (b) is performed using a microfluidic system employing flow-focusing geometry.

29. The method of claim 16, wherein said assessing is performed using a microfluidic system employing flow-focusing geometry.

30. The method of claim 17, wherein said isolating is performed using a microfluidic system employing flow-focusing geometry.
